(19) United States
(12) Patent Application Publication
(10) Pub. No.: US 2025/0349387 A1
(43) Pub. Date: Nov. 13, 2025

(54) FRAGMENTATION PATTERNS FOR AGING

(71) Applicant: Centre for Novostics, Shatin (HK)

(72) Inventors: Yuk-Ming Dennis Lo, Kowloon (CN); Kwan Chee Chan, Kowloon (CN); Peiyong Jiang, New Territories (CN); Guanhua Zhu, New Territories (CN); Wenlei Peng, New Territories (CN); Ruilong Zhou, New Territories (CN)

(21) Appl. No.: 19/202,718

(22) Filed: May 8, 2025

Related U.S. Application Data
(60) Provisional application No. 63/644,406, filed on May 8, 2024.

(51) Int. Cl. G16B 30/10; G16B 20/20; G16B 35/10; G16B 40/10; G16B 40/20; G16H 50/20

(52) U.S. Cl. CPC G16B 30/10; G16B 20/20; G16B 35/10; G16B 40/10; G16B 40/20; G16H 50/20

(57) ABSTRACT

The present disclosure describes techniques for predicting biological age based on fragmentomic patterns in cell-free DNA (cfDNA). In some examples, the techniques may include determining relative frequencies of sequence end motifs of cfDNA fragments, relative frequencies of cfDNA fragments of different, or a combination thereof for a biological sample from a subject. The relative frequencies can be used for predicting a biological age of the subject. For example, a feature vector can be generated using the relative frequencies of end motifs or the relative frequencies of the cfDNA fragments of each size. The feature vector can be input into a machine learning model trained using training samples having known chronological ages and having measured reference vectors of the end motifs or the sizes. The machine learning model may then be used to predict a biological age of the subject.

### CROSS-REFERENCES TO RELATED APPLICATIONS

The present application claims priority from and is a non-provisional application of U.S. Provisional Application No. 63/644,406, entitled “Fragmentation Patterns For Aging” filed May 8, 2024, the entire contents of which are herein incorporated by reference for all purposes.

### BACKGROUND

Ageing refers to the gradual physiological changes that occur in an organism over time (i.e., chronological age). The physiological changes may lead to senescence, a decline in biological functions and/or a decline in an organism's ability to adapt to metabolic stress. The metabolic stress can be driven by metabolic disturbances which are influenced by environmental factors such as pathogens, temperature, noise, toxins, nutrient imbalances (excess or deficiency), oxidative stress, and hypoxia. Ageing is a leading cause of disease and disability. Chronological age can be a risk factor for various diseases in the human population, such as cardiovascular diseases, diabetes, cancer, Alzheimer's disease, and dementia (Partridge et al., 2018). However, predictive power for a certain disease (e.g., Alzheimer's disease, cancers, cardiovascular diseases, etc.) can be low (Lowsky et al., 2014). And performing such predictions has a complexity far beyond what a person can perform mentally or with pen or paper and thus there have been limited development. Therefore, it would be beneficial to have improved techniques.

### BRIEF SUMMARY

The present disclosure describes techniques for predicting biological age based on fragmentomic patterns in cell-free DNA (cfDNA). In some examples, the techniques may include measuring quantities (e.g., relative frequencies) of sequence end motifs of cfDNA fragments, measuring sizes of cell-free DNA fragments, or a combination thereof for a biological sample from a subject. The quantities of sequence end motifs, the cfDNA fragment sizes, or the combination thereof can be used for predicting a biological age of the subject and/or for determining a presence of a pathology (e.g., a condition or disorder) in the subject. For example, one or more machine learning models can be trained to predict a biological age based on the relative frequencies of a set sequence end motifs in cfDNA fragments. Additionally or alternatively, the machine learning models can be trained to predict a biological age based on cfDNA fragment sizes. The machine learning models may be trained using sequencing data for subjects of various ages and with known disease statuses.

Additionally, a comparison of predicted biological age to chronological age of a subject can be used to detect a presence of a disorder in the subject. For example, a predicted biological age that exceeds (e.g., greater than or is less than) a chronological age by at least a threshold amount (e.g., age acceleration or deceleration) of the subject can be detected based on the comparison. A level of age acceleration can be used to classify the presence of a disorder. When the presence of a disorder is detected, a pathology (e.g., a particular condition or disorder) may be ascertained based on the particular tissue exhibiting age acceleration or based on the fragmentomic patterns analyzed. Accordingly, embodiments can provide measurements to inform physiological alterations, including cancers, autoimmune diseases, transplantation, and pregnancy.

In one embodiment, a method for measuring a biological age of a subject is provided. A computer system can perform the method. The computer system can receive sequence reads including ending sequences corresponding to ends of a plurality of cell-free DNA fragments from a biological sample of the subject. Additionally, the computer system can, for each of the plurality of cell-free DNA fragments, determine a sequence motif for each of one or more ending sequences of the cell-free DNA fragment. The computer system can also determine N relative frequencies of a set of N sequence motifs corresponding to the one or more ending sequences of the plurality of cell-free DNA fragments. N may be an integer equal to or greater than 16. The computer system can generate a feature vector using the N relative frequencies. The computer system can load a machine learning model into memory of the computer system. The machine learning model may be trained using training samples having known chronological ages and having measured reference vectors of the set of N sequence motifs of cell-free DNA fragments. Moreover, the computer system can input the feature vector into the machine learning model. The computer system can predict, using the machine learning model, the biological age of the subject.

In another embodiment, a method for measuring a biological age of a subject is provided. A computer system can perform the method. The computer system can receive sizes measured for a plurality of cell-free DNA fragments from a biological sample of the subject. Additionally, the computer system can, for each size of M sizes, determine a relative frequency of cell-free DNA fragments having that size. The computer system can generate a feature vector using the M relative frequencies. The computer system can loading a machine learning model into memory of the computer system. The machine learning model may be trained using training samples having known chronological ages and measured reference vectors of relative frequencies of the M sizes. Moreover, the computer system can input the feature vector into the machine learning model. The computer system can predict, using the machine learning model, the biological age of the subject.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1A shows a bar chart of age distributions of control subjects from dataset A.

FIG. 1B shows a bar chart of age distributions of control subjects from dataset B.

FIG. 1C shows a bar chart of age distributions of control subjects from dataset C.

FIG. 2A shows a plot of biological ages predicted based on 4-mer end motifs in cell-free DNA (cfDNA) fragments against true chronological ages for dataset A.

FIG. 2B shows a plot of biological ages predicted based on 4-mer end motifs in cell-free DNA (cfDNA) fragments against true chronological ages for dataset B.

FIG. 2C shows a plot of biological ages predicted based on 4-mer end motifs in cell-free DNA (cfDNA) fragments against true chronological ages for dataset C.

FIG. 3 shows a plot of biological ages predicted based on 3-mer end motifs in cfDNA fragments against true chronological ages.

FIG. 4 is a flowchart illustrating a method for measuring a biological age of a subject, according to some embodiments of the present disclosure.

FIG. 5A shows a plot of biological ages predicted based on cfDNA fragment sizes against true chronical ages for dataset A.

FIG. 5B shows a plot of biological ages predicted based on cfDNA fragment sizes against true chronical ages for dataset B.

FIG. 5C shows a plot of biological ages predicted based on cfDNA fragment sizes against true chronical ages for dataset C.

FIG. 6 is a flowchart illustrating a method for measuring a biological age of a subject, according to some embodiments of the present disclosure.

FIG. 7 shows a plot of end motif frequency against fragment size for cfDNA fragments, according to some embodiments of the present disclosure.

FIG. 8A shows a plot of predicted biological ages based on end motif frequencies and cfDNA fragment sizes against true chronological ages for dataset A.

FIG. 8B shows a plot of predicted biological ages based on end motif frequencies and cfDNA fragment sizes against true chronological ages for dataset B.

FIG. 8C shows a plot of predicted biological ages based on end motif frequencies and cfDNA fragment sizes against true chronological ages for dataset C.

FIG. 9 shows a plot of Pearson correlation values for an end motif clock, a size clock, and a fragmentomic clock combining motif and size.

FIG. 10 is a flowchart illustrating a method for measuring a biological age of a subject, according to some embodiments of the present disclosure.

FIG. 11 illustrates a system according to an embodiment of the present invention.

FIG. 12 shows a block diagram of an example computer system usable with system and methods according to some embodiments of the present disclosure.

FIG. 13 shows examples for end motifs according to some embodiments of the present disclosure.

### TERMS

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal dialysate, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

“Nucleic acid” may refer to deoxyribonucleotides or ribonucleotides and polymers thereof in either single- or double-stranded form. The term may encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs may include, without limitation, phosphorothioates, phosphoramidites, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “nucleotide,” in addition to referring to the naturally occurring ribonucleotide or deoxyribonucleotide monomers, may be understood to refer to related structural variants thereof, including derivatives and analogs, that are functionally equivalent with respect to the particular context in which the nucleotide is being used (e.g., hybridization to a complementary base), unless the context clearly indicates otherwise.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

“Single-molecule sequencing” refers to sequencing of a single template DNA molecule to obtain a sequence read without the need to interpret base sequence information from clonal copies of a template DNA molecule. The single-molecule sequencing may sequence the entire molecule or only part of the DNA molecule. A majority of the DNA molecule may be sequenced, e.g., greater than 50%, 55%, 60%, 65%, 70%, 75%, 80%, 85%, 90%, 95%, or 99%. A sequence read (or reads from both ends) can be aligned to a reference genome. When both ends are aligned (e.g., as part of a read of the entire fragment or for paired-ends), greater accuracy can be achieved in the alignment and a length of the fragment can be obtained. Embodiments of the present disclosure can use single-molecule sequencing.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location. The alignment procedure can be performed using various software packages, such as BLAST, FASTA, Bowtie, BWA, BFAST, SHRiMP, SSAHA2, NovoAlign and SOAP.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can be at least 1,000, 10,000, 50,000, 100,000, 500,000, 1,000,000, 5,000,000, 10,000,000, 50,000,000, 100,000,000, 500,000,000, one billion, or 3 billion nucleotides long, e.g., a full human genome or a repeat masked human genome. A reference may also include information regarding variations of the reference known to be found in a population of organisms.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment (e.g., 5′ end of either strand), and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 20, 30, 40, 50 60, 70, 80, 90, 100, 150, 200, 250, or 256 end motifs.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

A “relative frequency” (also referred to just as “frequency”) may refer to a relative value of one amount determined from nucleic acid fragments having a particular characteristic (e.g., an end motif or a size, such as a specified length) to one or more other amounts determined from nucleic acid fragments having a different characteristic. Examples include a ranking or a proportion (e.g., a percentage, fraction (ratio), or concentration). For example, a relative frequency of a particular end motif (e.g., A, CG, TAG, etc.) or end motif pair (e.g., A< >A) can provide a proportion of cell-free DNA fragments that have that end motif or that particular pair end motif pair. Such a proportion can be out of all the end motifs for a set of DNA molecules. As another example, the proportion can be a ratio of an amount for a particular end motif (or pair) relative to an amount of one or more other end motifs. As other examples, the relative frequency can be a ranking of amounts, e.g., raw counts of end motifs. The ranking can be of proportions (ratios) for each end motifs, as another example. Similar relative frequencies can be determined for size.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein can refer to a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio. A separation value can be compared to a threshold to determine whether the separation between the two values is statistically significant.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The phrase “healthy,” as used herein, generally refers to a subject possessing good health. Such a subject demonstrates an absence of any malignant or non-malignant disease. A “healthy individual” may have other diseases or conditions, unrelated to the condition being assayed, that may normally not be considered “healthy”.

The terms “cancer” or “tumor” may be used interchangeably and generally refer to an abnormal mass of tissue wherein the growth of the mass surpasses and is not coordinated with the growth of normal tissue. A cancer or tumor may be defined as “benign” or “malignant” depending on the following characteristics: degree of cellular differentiation including morphology and functionality, rate of growth, local invasion, and metastasis. A “benign” tumor is generally well differentiated, has characteristically slower growth than a malignant tumor, and remains localized to the site of origin. In addition, a benign tumor does not have the capacity to infiltrate, invade, or metastasize to distant sites. A “malignant” tumor is generally poorly differentiated (anaplasia), has characteristically rapid growth accompanied by progressive infiltration, invasion, and destruction of the surrounding tissue. Furthermore, a malignant tumor has the capacity to metastasize to distant sites. “Stage” can be used to describe how advance a malignant tumor is. Early stage cancer or malignancy is associated with less tumor burden in the body, generally with less symptoms, with better prognosis, and with better treatment outcome than a late stage malignancy. Late or advanced stage cancer or malignancy is often associated with distant metastases and/or lymphatic spread.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g. recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g. symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “level of pathology” (also referred to as a condition) can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology.

A “biological age” can refer to a measure of a state of an aging process of a subject. A biological age can reflect how well cells and tissues are functioning as compared to an expectation of the functioning of the cells and tissues based on a chronological age (e.g., a simple count of years since birth) of the subject. In contrast to chronological age, biological age may indicate an impact of genetics, lifestyle, and environmental factors on a subject's aging process, vitality, and resilience.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, one million, ten million, 100 million, or one billion parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network (e.g. including convolutional and/or transformer layers) that may have 1-10 layers as examples, recurrent neural network (e.g., long short term memory, LSTM), boosting (meta-algorithm), bootstrap aggregating (bagging) such as random forests, support vector machine (SVM), support vector (SVR), Bayesian statistics, case-based reasoning, decision tree learning, inductive logic programming, linear regression, logistic regression, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), ordinal classification, data pre-processing, handling imbalanced datasets, statistical relational learning, or Proaftn (a multicriteria classification algorithm), or an ensemble of any of these types. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Cell-free DNA (cfDNA) can occur naturally in the form of short fragments in various types of biological samples, such as in plasma, urine, saliva, cerebrospinal fluid, pleural fluid, amniotic fluid, peritoneal fluid, and ascitic fluid. In contrast to DNA contained in a particular tissue, plasma or other biological samples can carry cfDNA molecules released from dying cells from various tissue. Thus, examination of cfDNA from biological samples can provide minimally invasive access to DNA molecules from various tissues. This can enable detection and analysis of abnormal or diseased tissue (e.g., organs).

To determine states of biological processes, approaches to analyze fragmentomic patterns of cfDNA (e.g., cfDNA fragment sizes, end motifs, or the combination thereof) can be developed. For example, sequence reads corresponding to ends of one or more cfDNA molecules from a subject can be aligned with a reference genome. One or more nucleotides of the reference genome corresponding to the end of the cfDNA molecules can be an end motif. Additionally or alternatively, a distance between each end of the cfDNA molecules can indicate the size of the cfDNA molecule. Thus, based on the sequence reads or based on aligning the sequence reads to the reference genome, the end motifs and/or the cfDNA molecule sizes can be identified.

Models can be developed for predicting the states of a biological process using fragmentomic patterns. For example, a machine learning model can be trained using fragmentomic patterns (e.g., end motif frequency or sizes) of cfDNA molecules from biological samples from subjects of varying age, disease status (e.g., subjects that have not been diagnosed with a particular disease or subjects diagnosed with a particular disease), or a combination thereof. In a particular example, a machine learning model can be trained using relative frequencies of particular end motifs of cfDNA molecules from subjects without a disease, such as without cancer. Such machine learning models can provide predictions that could not be practically provided by a person mentally or with pen and paper.

In another example, a machine learning model can be trained using relative frequencies of cfDNA molecules of certain sizes, in which the cfDNA molecules may also be obtained from subjects without the disease. Additionally, a machine learning model can be trained using relative frequencies of end motifs for cfDNA molecules of certain sizes. As a result of training, the machine learning models may output a predicted biological age based on receiving input with the relative frequencies of end motifs, the relative frequencies of cfDNA molecules of certain sizes, or the relative frequencies of end motifs per size for a biological sample. Thus, the machine learning model can utilize fragmentomic patterns to predict the biological age of a subject.

In some examples, the predicted biological age output by the machine learning model for a subject can be compared to a true chronological age of the subject to reveal age aberrations (e.g., age acceleration or age deceleration). Age aberrations can be indicative of a health issue for the subject, such as a presence of a condition, disease, or disorder. For example, a presence or progression of one or more diseases can be identified based on the difference between a predicted biological age and a true chronological age.

As a result of analyzing fragmentomic patterns for cfDNA and developing approaches to predict age, disease occurrence, or disease progression based on fragmentomic patterns, a deeper understanding of related biological processes can be achieved. For example, a deeper understanding of an impact of diseases on particular organs or of effects of aging can be obtained. This can facilitate development of methods for effective detection and treatment of diseases. For example, the ageing assessment based on fragmentomic patterns can enable disease detection in a minimally invasive manner, which can lead to development of novel preventative interventions.

### I. BIOLOGICAL AGE

Biological age can reflect how old an organism is based on physiological or molecular evidence. Biological age can be associated with age-related biological processes and pathophysiological states. For example, if a subject is especially healthy, the subject's biological age may be lower than the subject's chronological age, which can be referred to as ‘decelerated biological ageing’. Otherwise, ‘accelerated biological ageing’ may be detected in subjects with immune-related and/or organ-related dysfunctions and can indicate a high risk of developing one or more illnesses. Hence, the determination of biological age can be important for preventive diagnosis and precision medicine. A standard curve between biological age and physiological or molecular evidence may be constructed from a population of defined control subjects, so that the biological age can be quantified for each testing sample. The control subjects can be defined as subjects that do not have the disease(s) or disorder(s) being interest during the period of investigation.

Recent advances in molecular biology and omics technologies have enabled the characterization of biological ageing at the molecular level and proposed some aging clocks for estimating human biological age. For example, based on DNA cytosine-phosphate-guanine (CpG) methylation, Hannum et al. predicted chronological age using blood samples (Hannum et al., 2013) and Horvath et al. built the pan-tissue methylation ageing clocks that apply to all human tissues (Horvath, 2013). Additionally, Peters et el. attempted to develop the ageing clocks using transcriptomic data from peripheral blood (Peters et al., 2015) and Fleischer et at. attempted to develop the ageing clocks using transcriptomic data from dermal fibroblasts (Fleischer et al., 2018). The use of metabolites in the urine (Hertel et al., 2016) and blood (Robinson et al., 2020) may also allow the development of metabolomic ageing clocks. But such approaches based on methylation or transcriptomic information are restricted to the intracellular level. In another example, Lehallier et al. used circulating proteins in plasma to predict chronological age (Lehallier et al., 2019) and Oh et al. demonstrated organ-specific proteomic ageing clocks in living individuals (Oh et al., 2023). There is a paucity of ageing clocks based on molecular information concerning cell-free DNA molecules.

In some aspects of the present disclosure, approaches to developing ageing clocks based on the fragmentomic patterns of cell-free DNA (cfDNA) are provided. CfDNA can be DNA fragments found in bodily fluids, such as plasma, cerebrospinal fluid, urine, bile, lymph, saliva, synovial fluid, serous fluid, pleural fluid, amniotic fluid, etc. CfDNA molecules are nonrandomly fragmented, thereby forming characteristic fragmentation patterns (i.e., ‘fragmentomics’). Characteristic fragmentation patterns can include fragment length, end motif, end jaggedness, and nucleosomal footprint.

Assessing age using cfDNA fragmentation patterns has advantages over the existing ageing clocks mentioned above. For example, compared with the use of cellular DNA-based clocks, the use of cfDNA can provide noninvasive access to clocks for any organ as cfDNA molecules in blood circulation can be released from any tissue. Additionally, fragmentomic features can be obtained from shallow sequencing that is cost-effective. Shallow sequencing can have whole-genome coverage ranging typically from ˜0.1× to ˜5× (e.g. less than or equal to 0.05×, 0.1×, 0.2×, 0.5×, 1×, 2×, 3×, 4×, 5×, 6×, 7×, 8× etc.). Thus, comparison between biological ages estimated using aging clocks that utilize fragmentomic patterns and true chronological ages can allow for the determination of the accelerated or decelerated aging in a biology sample in a cost-effective manner. This, in turn, provides an opportunity to inform, prevent, and treat diseases effectively.

### II. COHORTS

Sequencing data (e.g., whole-genome or targeted sequencing) can be used in some embodiments of the present disclosure to develop machine learning models for predicting biological age. For example, a first dataset (dataset A), a second dataset (dataset B), and a third dataset (dataset C), can include whole-genome paired-end sequencing data for control subjects (e.g., subjects without cancer). The whole-genome paired-end sequencing data of the datasets is shallow sequencing data (<5×). The datasets can further include chronological ages for each of the control subjects.

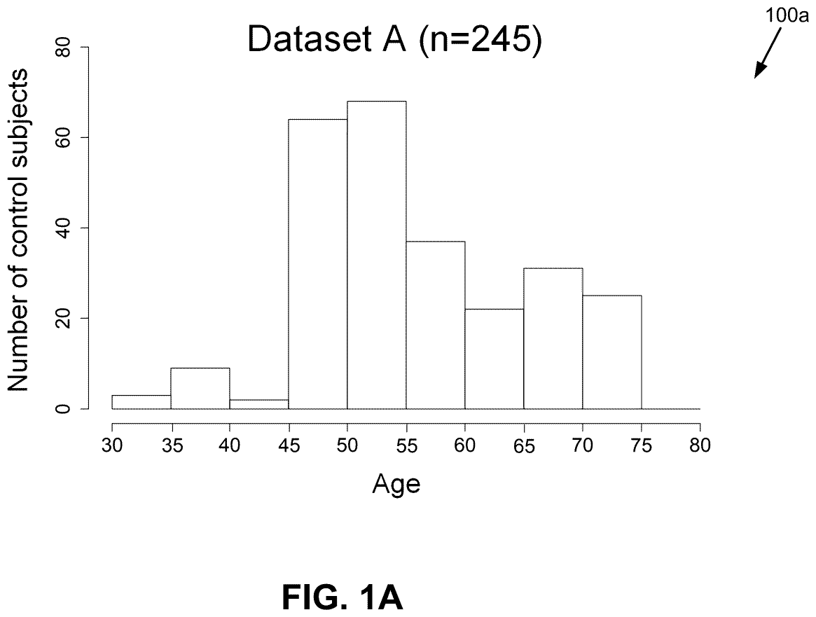

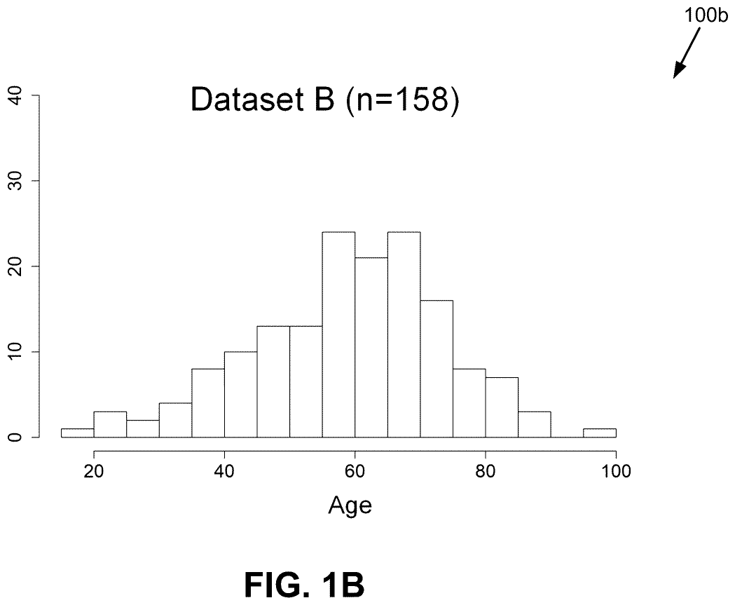

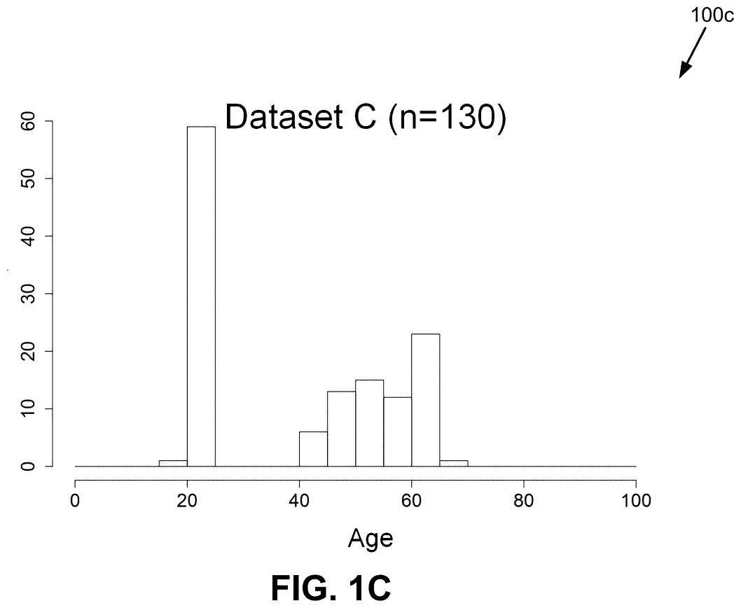

FIGS. 1A-1C show bar charts 100a-c of age distributions of the control subjects in each dataset. As shown in plot 100a of FIG. 1A, an age range of the 245 control subjects in dataset A spans from thirty-four to seventy-five. Additionally, as shown in plot 100b of FIG. 1B, an age range of the 158 control subjects in dataset B spans from nineteen to ninety-six. As shown in plot 100c of FIG. 1C, an age range of the 130 control subjects in dataset C spans from twenty to sixty-six. These datasets are referenced below as results are provided for various techniques according to embodiments of the present disclosure.

### III. BIOLOGICAL AGE PREDICTION BASED ON END MOTIFS

An end motif can relate to an ending sequence of a cell-free DNA (cfDNA) fragment. That is, the end motif can be the sequence of N bases at a 5′ end of either strand (Watson or Crick) of a cfDNA fragment. In some examples, the ending sequence corresponding to an end motif can be a K-mer ending sequence having “K” number of bases (e.g., 1, 2, 3, 4, 5, 6, 7, 8, 9, 10, 11, 12, 13, 14, 15, etc. bases). The end motif (or “sequence motif”) can relate to the sequence itself rather than to a particular position in a reference genome. Thus, a particular end motif may occur at numerous positions throughout a reference genome.

The end motif may be determined using a reference genome (e.g., based on alignment of a sequence read to the reference genome) or determined from just the sequence itself. For example, the end motif can be determined using the outermost nucleotides of a sequence read or by aligning one or more sequence reads corresponding to one or more cfDNA fragments to a reference genome. For instance, the N bases before an end position (last N bases of a DNA fragment) or just after a start position (first N positions of a DNA fragment) can be identified.

Some or all of the end motifs of a set (i.e., corresponding to the value of K) can be used. For example, all of the 256 end motifs for 4-mers can be used, or only certain 4-mers can be used.

### A. Various Types of End Motifs

As mentioned above, an end motif relates to the ending sequence of a cell-free DNA fragment, e.g., the sequence for the K bases at either end of the fragment. Additionally, as mentioned above, the ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

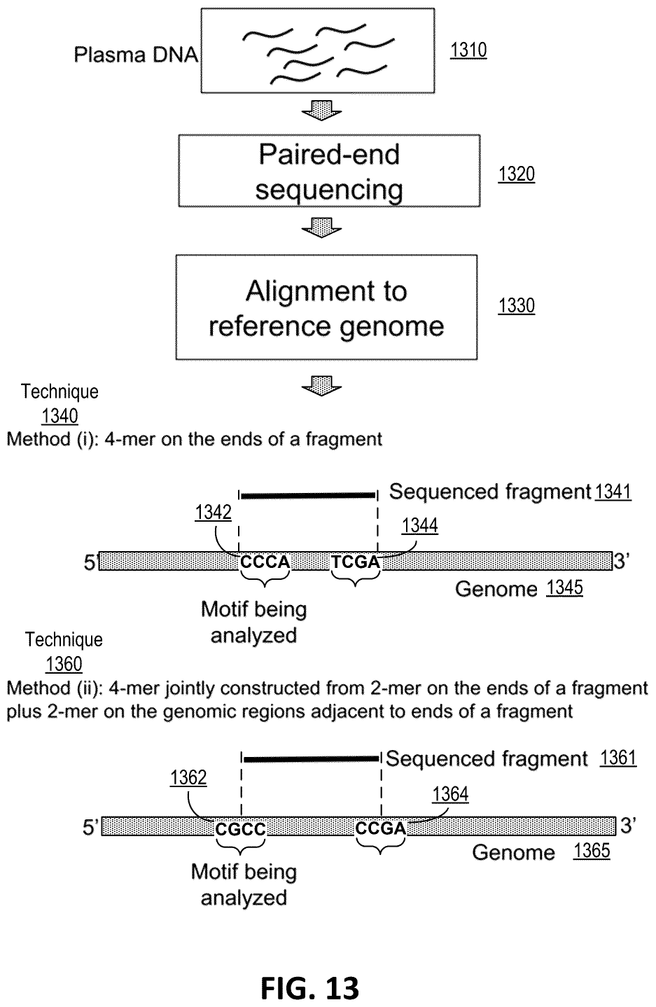

FIG. 13 shows examples for end motifs according to embodiments of the present disclosure. FIG. 13 depicts two ways to define 4-mer end motifs to be analyzed. In technique 1340, the 4-mer end motifs are directly constructed from the first 4-bp sequence on each end of a plasma DNA molecule. For example, the first 4 nucleotides or the last 4 nucleotides of a sequenced fragment could be used. In technique 1360, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 13, cell-free DNA fragments 1310 are obtained, e.g., using a purification process on a blood sample, such as by centrifuging. Besides plasma DNA fragments, other types of cell-free DNA molecules can be used, e.g., from serum, urine, saliva, and other mentions herein. In one embodiment, the DNA fragments may be blunt-ended.

At block 1320, the DNA fragments are subjected to paired-end sequencing. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment.

At block 1330, the sequence reads can be aligned to a reference genome. This alignment is to illustrate different ways to define a sequence motif, and may not be used in some embodiments. The alignment procedure can be performed using various software packages, such as BLAST, FASTA, Bowtie, BWA, BFAST, SHRiMP, SSAHA2, NovoAlign and SOAP.

Technique 1340 shows a sequence read of a sequenced fragment 1341, with an alignment to a genome 145. With the 5′ end viewed as the start, a first end motif 1342 (CCCA) is at the start of sequenced fragment 1341. A second end motif 1344 (TCGA) is at the tail of the sequenced fragment 1341. When analyzing the end predominance of a cell-free DNA (cfDNA) fragments (e.g., plasma DNA), this sequence read would contribute to a C-end count for the 5′ end. Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A.

Technique 1360 shows a sequence read of a sequenced fragment 1361, with an alignment to a genome 1365. With the 5′ end viewed as the start, a first end motif 1362 (CGCC) has a first portion (CG) that occurs just before the start of sequenced fragment 1361 and a second portion (CC) that is part of the ending sequence for the start of sequenced fragment 1361. A second end motif 1364 (CCGA) has a first portion (GA) that occurs just after the tail of sequenced fragment 1361 and a second portion (CC) that is part of the ending sequence for the tail of sequenced fragment 1361. Such end motifs might, in one embodiment, occur when an enzyme recognizes CGCC and then makes a cut just before the G and the C. If that is the case, CC will preferentially be at the end of the plasma DNA fragment with CG occurring just before it, thereby providing an end motif of CGCC. As for the second end motif 1364 (CCGA), an enzyme can cut between C and G. If that is the case, CC will preferentially be at the end of the plasma DNA fragment. For technique 1360, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end motif, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 1360 makes an association of an ending sequence to other bases, where the reference is used as a mechanism to make that association. A difference between techniques 1340 and 1360 would be to which two end motif a particular DNA fragment is assigned, which affects the particular values for the relative frequencies. But the overall result (e.g., fractional concentration of clinically-relevant DNA, classification of a level of pathology, etc.) would not be affected by how a DNA fragment is assigned to an end motif, as long as a consistent technique is used for the training data as used in production.

The counted numbers of DNA fragments having an ending sequence corresponding to a particular end motif may be counted (e.g., stored in an array in memory) to determine relative frequencies. As described in more detail below, a relative frequency of end motifs for cell-free DNA fragments can be analyzed. Differences in relative frequencies of end motifs have been detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

Examples use of 5′ end motifs. In some embodiments, single strand assay techniques can be used to obtain information for both strands at the 5′ ends. A single-stranded library preparation can be used. For example, commercialized kits for ssDNA library preparation can include, but not limited to, xGen™ ssDNA & Low-Input DNA Library Preparation Kit (IDT®), VAHTS ssDNA Library Prep Kit (Vazyme®), ssDNA Library Prep Kit (iGeneTech®), and XACTLY or SRSLY Kits for NGS (CLARETBIO®).

### B. End Motif Clock

In some embodiments, end motif patterns (e.g., relative frequencies of end motifs) in cfDNA can be analysed and used for age prediction based on various techniques. A feature vector can be generated using the relative frequencies of end motifs end. Such a feature vector can provide a fragmentation pattern of end motifs. A machine learning model can then process the feature vector. Examples of machine learning models that may be used for age prediction based on end motif patterns can include absolute shrinkage and selection operator (LASSO), ridge regression, support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm, etc.

As further examples, a model (e.g., a machine learning model) may utilize linear regression, logistic regression, a deep recurrent neural network (e.g., long short-term memory, etc.), a hidden Markov model (HMM), linear discriminant analysis (LDA), k-means clustering, density-based spatial clustering of applications with noise (DBSCAN), random forest algorithm, etc. to predict age based on end motif patterns. Such a model for predicting age based on end motifs can be referred to herein as an end motif clock.

In a particular example, a model (e.g., a LASSO regression model) can be developed for predicting biological age based on the relative frequencies of each 4-mer end motifs, although other lengths of end motifs can be used. The 4-mer end motifs can be ending sequence that include any combination of the four nucleotide bases (e.g., adenine (A), thymine (T), cytosine (C), and guanine (G)). Thus, examples of the 4-mer end motifs can be include ATCG, TTTT, GCGC, etc.

The 4-mer end motifs of cfDNA fragments can be determined from any suitable assay, e.g., using sequencing or probe-based technique. In one example, whole-genome paired-end sequencing data for control subjects in dataset A, dataset B, and dataset C respectively can be used. The 4-mer end motifs can be determined using the first 4-nucleotide (i.e., 4-mer) sequence on each 5′ fragment end with reference to a human reference genome. The first 4-nucleotide sequence on each 5′ fragment end can be referred to as a 5′ 4-mer end motif Sequence reads (e.g., the paired-end sequencing data) for cfDNA fragments for each control subject in each dataset can be aligned to a reference genome (e.g., a human reference genome).

When alignment is performed, the sequencing reads of the paired-end sequencing data can be aligned to the reference genome, the smallest coordinate on the reference genome for each sequencing read can be defined as the 5′ end. Typically, a 4-mer end motif at a 5′ end of a cfDNA fragment can match a corresponding four nucleotides in the reference genome (e.g., the nucleotides on the Watson strand of the reference genome). Alternatively, in some examples, the 5′ 4-mer end motif of a sequence read can be derived from the Crick strand.

Therefore, the end motif clock can be established using 4-mer end motifs for the 5′ end of each cfDNA fragment as derived from the Watson strand, the Crick strand, or a combination thereof. In other embodiments, the end motifs of cfDNA fragments used for the end motif clock can be the first 1-, 2-, 3-, 4-, 5-, 6-, 7-, 8-, 9-, 10, or more nucleotides (or other number mentioned herein) on the 5′ end of cfDNA fragments.

After each end motif (e.g., 5′ 4-mer end motif) for each cfDNA fragment is determined, a relative frequency of each 5′ 4-mer end motif for cfDNA fragment can be determined. The relative frequency can be a proportion of cfDNA fragments corresponding to the sequencing data (e.g., whole-genome paired-end sequencing data) for each control subject in each dataset that have each 5′ 4-mer end motif. If ending sequences of both ends of a fragment are determined, the proportion would be out of all of the ending sequences. For example, if CCCA occurred in 100 of the ending sequences out of the 10,000 ending sequences obtained from both ends of 5,000 cfDNA molecules, then the proportion (example of a relative frequency) would be 0.01 or 1%.

In some examples, a ranking of each end motif (e.g., each 5′ 4-mer end motif) can be determined based on the amounts (e.g., raw count or relative frequency) of each end motif. For example, the end motifs can be ranked from an end motif with a highest amount to an end motif with a lowest amount or vice versa. Thus, the ranking can be representative of a level of the amount of each end motif with respect to the remaining end motifs of the cfDNA fragments. As the ranking is determined based on the relative amounts (e.g., raw counts or proportion of end motifs), the ranking is a type of relative frequency. For instance, the end motif of CCCA being ranked 4^{th} is a relative frequency compared to end motif CCGA ranked 8^{th} in that CCCA would then occur more frequently than CCGA.

Additionally or alternatively, a ratio of the amounts (e.g., proportion, rankings, or raw counts) of each end motif with respect to the amounts of one or more other end motifs can be determined. As can be done when the relative frequency is a proportion out of all of a set of ending sequences, the techniques using a ranking and such a ratio as a relative frequency can be determined from a send of ending sequences when ending sequences of both ends are used.

Once the relative frequencies (e.g., proportion, ranking, or ratio) are determined using each end motif of each cfDNA fragment, the control subjects for each dataset can be split for training and testing with a ratio of, for example, 4:1. As a result, a training dataset can comprise relative frequencies and/or the other suitable parameters of each 5′ 4-mer end motif for control subjects for training, and a testing dataset can comprise relative frequencies and/or the other suitable parameters of each 5′ 4-mer end motif for control subjects for testing.

The model can then be trained and verified using the training dataset and the testing dataset for each of dataset A, B, and C respectively. The training can include fitting the model to the training dataset. That is, training can include tuning parameters and possibly hyperparameters associated with the model to improve age prediction by the model based on the relative frequencies of 5′ 4-mer end motifs in the training dataset. As will be appreciated by the skilled person, various training techniques can be used to optimize the parameters to fit the model to the training dataset.

After training, the model can be tested by inputting 5′ 4-mer end motif relative frequencies from the testing dataset into the trained model. The trained model can then output predicted biological ages for the control subjects in the testing dataset based on the relative frequencies of the 5′ 4-mer end motifs. The predicted biological ages can then be compared to true chronological ages of the subjects to estimate an accuracy of the trained model.

### 1. Results Using 4-Mer End Motifs

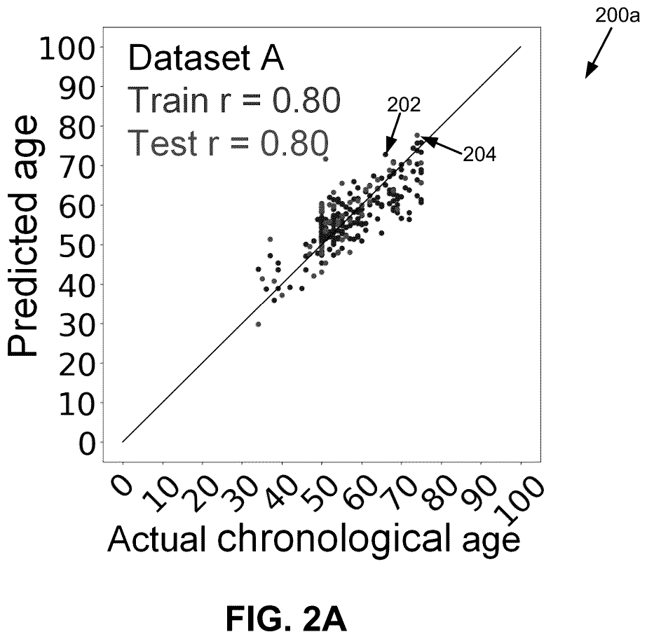

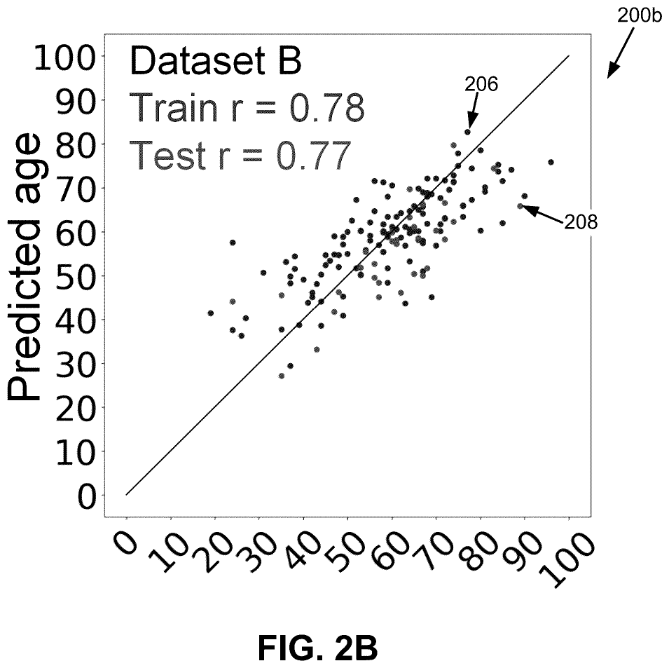

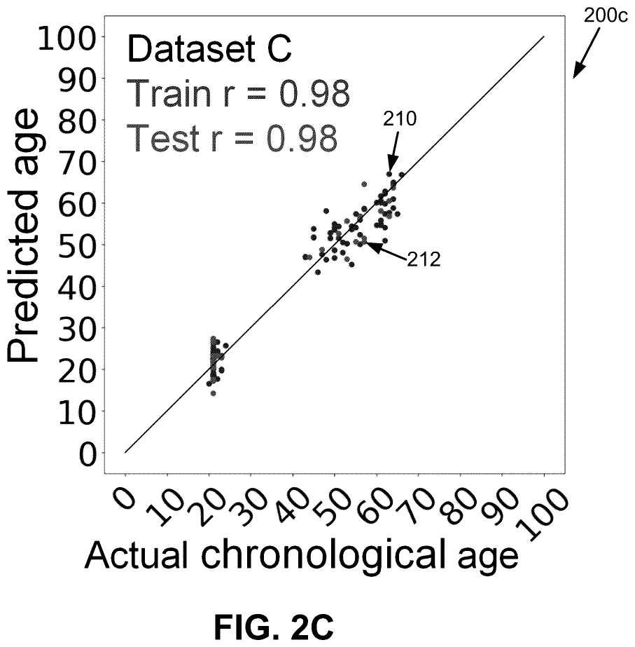

FIG. 2A shows a plot 200a of biological ages predicted based on 5′ 4-mer end motifs in cell-free DNA (cfDNA) fragments against true chronological ages for dataset A. In particular, —plot 200a shows the biological age predictions output by the trained model based on the 5′ 4-mer end motifs for control subjects in the training dataset and the testing dataset associated with dataset A. As an example, point 202 shows a predicted biological age for a subject in the training data set plotted against a true chronological age of the subject, while point 204 shows a predicted biological age for a subject in the testing data set plotted against a true chronological age of the subject. The accuracy for the training data set and the testing data set is the same, r=0.80.

FIG. 2B shows a plot 200b of biological ages predicted based on 5′4-mer end motifs in cell-free DNA (cfDNA) fragments against true chronological ages for dataset B. In particular, plot 200b shows the biological age predictions output by the trained model based on the 5′ 4-mer end motifs for the control subjects in training dataset and the testing dataset associated with dataset B. As an example, point 206 shows a predicted biological age for a subject in the training data set plotted against a true chronological age of the subject, while point 208 shows a predicted biological age for a subject in the testing data set plotted against a true chronological age of the subject. The accuracy for the training data set is r=0.78 and the testing data set is r=0.77.

FIG. 2C shows a plot 200c of biological ages predicted based on 4-mer end motifs in cell-free DNA (cfDNA) fragments against true chronological ages for dataset C. In particular, plot 200c shows the biological age predictions output by the trained model based on the 5′ 4-mer end motifs for the control subjects in the training dataset and the testing dataset associated with dataset C. As an example, point 210 shows a predicted biological age for a subject in the training data set plotted against a true chronological age of the subject, while point 212 shows a predicted biological age for a subject in the testing data set plotted against a true chronological age of the subject. The accuracy for the training data set is r=0.98 and the testing data set is r=0.98.

Each of the plots 200a-c shows that the predicted biological ages output by the trained model can be highly correlated with the actual chronological ages of the control subjects in datasets A, B, and C respectively. To quantify the correlation between the predicted and true ages depicted in FIGS. 2A-C, a Pearson's correlation coefficient was e computed for each training dataset and each testing dataset. For example, the Pearson's correlation coefficient for the training dataset of dataset A is 0.80 with a p value of less than 0.001 and the Pearson's correlation coefficient for the testing dataset of dataset A is 0.80 with a p value of less than 0.001. Additionally, the Pearson's correlation coefficient for the training dataset of Dataset B is 0.78 and the Pearson's correlation coefficient for the testing dataset of dataset B is 0.77. The p value for dataset B is also 0.001. Moreover, the Pearson's correlation coefficient for the training dataset and the testing dataset of dataset C is 0.98 with a p value of less than 0.001. Thus, in the example, a high concordance between the ages predicted by the end motif clock (e.g., the trained model) and the chronological ages in datasets A, B, and C was found.

### 2. Results Using 3-Mer End Motifs

In another example, a model (e.g., a LASSO regression model) can be developed for predicting biological age based on the relative frequencies of each 3-mer end motifs. The 3-mer end motifs can be an ending sequence that includes any combination of three of the nucleotide bases. Thus, examples of the 3-mer end motifs can be include ATA, CGC, TGG, etc.

The 3-mer end motifs of cfDNA fragments can be determined in a similar manner as the 4-mer end motifs, e.g., from the whole-genome paired-end sequencing data in Dataset A. The 3-mer end motifs can be determined using the first 3-nucleotide (i.e., 3-mer) sequence on each 5′ fragment end with reference to a human reference genome. Similar to the previous example, the sequence reads (e.g., the paired-end sequencing data) for cfDNA fragments for each control subject in Dataset A can be aligned to the human reference genome. The smallest coordinate on the reference genome for each sequencing read can be defined as the 5′ end. Due to the 3-mer end motifs used in the example being on the 5′ fragment end, the 3-mer end motifs may be determined based on the first three nucleotides on the Watson strand of the reference genome from the smallest coordinate associated with each read. Alternatively or additionally, the 5′ 3-mer end motifs can derived from the Crick strand.

After each 5′ 3-mer end motif for each cfDNA fragment is determined, a relative frequency of each 5′ 3-mer end motif can be determined. Once the relative frequencies are determined, the control subjects of Dataset A can be split for training and testing with a ratio of, for example, 4:1. As a result, a training dataset can comprise relative frequencies of each 5′ 3-mer end motif for control subjects for training and a testing dataset can comprise relative frequencies of each 5′ 3-mer end motif for control subjects for testing.

The model can then be trained and verified using the training dataset and the testing dataset respectively. The training can include fitting the model to the training dataset. The verifying can include inputting the 5′ 3-mer end motif relative frequencies from the testing dataset into the trained model. The trained model can output predicted biological ages for the control subjects in the testing dataset based on the relative frequencies of the 5′ 3-mer end motifs. The predicted biological ages can then be compared to the true chronological ages of the control subjects in the testing dataset to estimate an accuracy of the trained model.

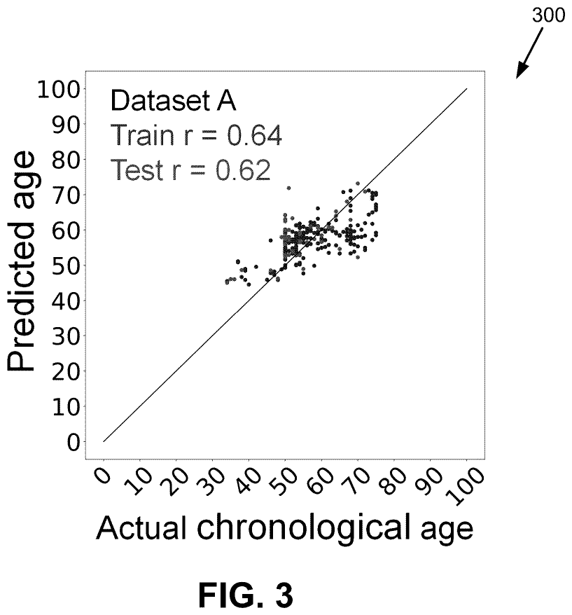

FIG. 3 shows a plot 300 of biological ages predicted based on 3-mer end motifs in cfDNA fragments against true chronological ages. In particular, plot 300 shows the biological age predictions output by the trained regression model based on the 5′ 3-mer end motifs for the control subjects in Dataset A. Additionally, as shown in FIG. 3, the Pearson's correlation coefficient for the training dataset of dataset A is 0.64 and the Pearson's correlation coefficient for testing dataset of dataset A is 0.62.

### C. Example Method for Age Prediction Using End Motifs

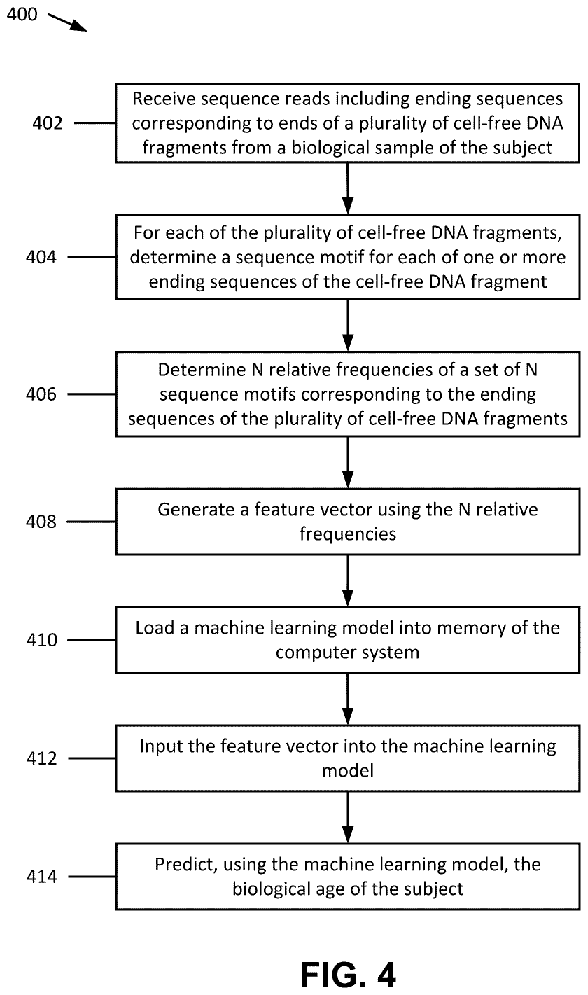

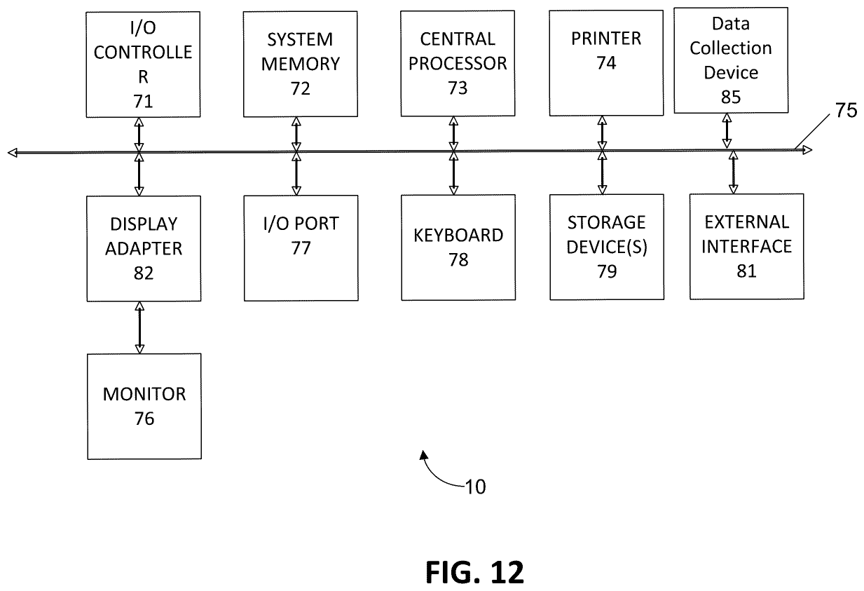

FIG. 4 is a flowchart illustrating a method 400 for measuring a biological age of a subject, according to some embodiments of the present disclosure. Portions or all steps of method 400 can be performed by a computer system (e.g., computer system 1200 shown in FIG. 12), including one or more processors. Method 400 can use a trained ML model that was trained by the computer system or another computer system. The computer system can comprise various devices, e.g., one device that performed the training and another device that uses the trained model.

At block 402, the method 400 can include receiving sequence reads including end sequences corresponding to ends of a plurality of cfDNA fragments from a biological sample of the subject. The biological sample can be any cell-free sample from the subject, e.g., as described herein, such as plasma, urine, saliva, cerebrospinal fluid, pleural fluid, amniotic fluid, peritoneal fluid, ascitic fluid, or the like. As examples, the sequence reads can be generated from paired-end sequencing, single-molecule sequencing, targeted sequencing, or the like, as well as probe-based techniques.

In some examples, prior to receiving the sequence reads, the method 400 can include analyzing the plurality of cfDNA fragments from the biological sample to obtain the sequence reads. The analysis can include detecting signals measured from the plurality of cfDNA fragments. For example, the sequence reads may be determined using sequencing or probe-based techniques, as may be done using a microarray or in an amplification reaction (e.g., PCR), performed on the biological sample from the subject. Additionally or alternatively, analyzing the plurality of cfDNA fragments can include preparing a sequencing library from the plurality of cfDNA fragments and sequencing the sequency library.

At block 404, the method 400 can include, for each of the plurality of cfDNA fragments, determining a sequence motif for each of one or more ending sequences of the cfDNA fragment. In doing so, a set of ending sequences for the plurality of cfDNA fragments is determined. Each sequence motif for each ending sequence in the set of ending sequences can include M base positions. In some examples, the sequence motif for one or more ending sequences for each cfDNA fragment can be directly identified from the sequencing reads. The first M bases from each sequence read (5′ end on one strand) or the reverse complement of the last M bases (5′ end on other strand) can be the sequence motif of the ending sequence of each cfDNA fragment. As examples, M can be at least 1, 2, 3, 4, 5, 6, or 7. In one implementation, M can be at least two.

In other examples, the sequence reads can be aligned to a reference genome. The alignment can provide genomic context for the cfDNA fragments. The alignment procedure can be performed using various software packages, such as BLAST, FASTA, Bowtie, BWA, BFAST, SHRiMP, SSAHA2, NovoAlign and SOAP. After alignment, similar to the above, the first M bases from each sequence read, the last M bases from each sequence read, or the reverse complement of the first or last M bases can be the sequence motif of the ending sequence of each cfDNA fragment. The first M bases or the last M bases can be identified based on positioning of the sequence read with respect to the reference genome. For example, the first M bases can start at a smallest coordinate on the reference genome corresponding to an aligned sequence read.

At block 406, the method 400 can include determining N relative frequencies of a set of N sequence motifs corresponding to the set of ending sequences of the plurality of cfDNA fragments. In various examples N can be at least 4, 5, 6, 7, 8, 9, 10, 11, 12, 13, 14, 15, 16, 32, 64, 70, 80, 90, 100, 110, 120, 140, 150, 160, 170, 180, 190, 200, 210, 220, 230, 240, 250, 256, or any integer there between. In one implementation, N can be an integer equal to or greater than 16.

A relative frequency of a sequence motif may include a proportion of the set of ending sequences corresponding to the sequence motif. Alternatively, a relative frequency of a sequence motif may include a ratio of (1) a first amount of the set of ending sequences corresponding to the sequence motif and (2) a second amount of the set of ending sequences that are different from the sequence motif Moreover, in another example, a relative frequency of a sequence motif includes a ranking of a first amount of the set of ending sequences that have the sequence motif relative to amounts of the set of ending sequences that have other sequence motifs different than the sequence motif.

At block 408, the method 400 can include generating a feature vector using the N relative frequencies. The feature vector can include the N relative frequencies of the set of N sequence motifs determined for the cfDNA fragments of the biological sample from the subject. The feature vector can include the N relative frequencies in a structured form that can be ingested (input) into and understood by a machine learning model. As examples, the feature vector can include at least 16, 32, 64, 128, 256, 1,024, and 4,096 features.

At block 410, the method 400 can include loading a machine learning model into memory of the computer system. The machine learning model can be trained using training samples having known chronological ages and measured reference vectors of the set of N sequence motifs of cfDNA fragments. For example, the training samples can be biological samples taken from one or more training subjects with known chronological ages. The relative frequences of the set of N sequence motifs can be measured from the training samples. The machine learning model can be a regression model or another suitable type of machine learning model. In an example, the training samples can be obtained from a training cohort, such as the cohorts (e.g., dataset A, dataset B, and dataset C) described herein. The training samples can be control subjects (e.g., one without cancer). The training cohort can include a known chronological age for each subject without cancer.

At block 412, the method 400 can include inputting the feature vector into the machine learning model. That is, the N relative frequencies of the set of N sequence motifs determined for the cfDNA fragments of the biological sample from the subject can be input into the machine learning model.

At block 414, the method 400 can include predicting, using the machine learning model, the biological age of the subject. As examples, the biological age predicted can be a year (e.g., 20, 30, 45, 55, etc.) or the biological age can be an age range (e.g., 20-25, 30-39, etc.), or even higher resolution than a year, e.g., a month of range of months. Additionally, in some examples, an output of the machine learning model can be a probability for each of a set of ages (e.g., 20, 25, 30, 35) or age ranges (e.g., 20-29, 30-39, etc.). In such examples, the biological age predicted by the machine learning model can be the age or age range with a highest probability. In some examples, the biological age predicted using the machine learning model can be compared to a true chronological age of the subject. If the predicted age deviates from the true chronological age, e.g. greater than the true chronological age by a threshold amount, the subject can be determined to have a pathology (e.g., a condition, disease or disorder). In such an instance, an alert or other suitable indicator of age acceleration can be generated and output.

In some examples, the method can include determining a separation value by comparing the predicted biological age to the true chronological age of the subject. A classification of a pathology for the subject can then be determined based on the separation value. For example, the separation value can be compared to one or more reference values determined from at least a first cohort of subjects that have a particular classification of the pathology and a second cohort of subjects that do not have the particular classification of the pathology. The particular classification can be (1) whether the pathology is present or (2) a severity or stage of the pathology. The pathology can be cancer or another suitable pathology (e.g., another condition, disease or disorder). The machine learning model may generate each reference value based on training samples from training subjects with the with the particular classification or without the particular classification.

A difference between the separation value and the one or more reference values can be determined. If the separation value sufficiently similar to the reference value (e.g., if the distance is within a threshold or is the closest reference value of more than one reference value), then the subject can be determined to have the particular classification corresponding to the reference value. For example, if the reference value is for subjects with the pathology and the difference is less than the threshold, the subject can be determined to have the particular classification of the pathology.

### IV. BIOLOGICAL AGE PREDICTION BASED ON FRAGMENT SIZE

A fragment size can relate to a number of base pairs (also referred to as bases for length of a single strand) that make up a cell-free DNA (cfDNA) fragment. CfDNA fragments can be relatively short. For example, a substantial portion of cfDNA fragments may be around 160-180 base pairs long. The size distribution (size profile) of cfDNA fragments can provide valuable insights into their cellular origins and the physiological or pathological processes (e.g., aging) occurring within a subject. Techniques such as next-generation sequencing, electrophoresis, or other bioanalytical platforms can be used to determine the fragment sizes.

### A. Fragment-Size Clock

In some embodiments, sizes of cfDNA fragments can be analysed and used for age prediction based on various techniques. A feature vector can be generated using the relative frequencies of cfDNA fragments of particular sizes or size ranges. Such a feature vector can provide a fragmentation pattern of sizes. A machine learning model can then process the feature vector. Examples of machine learning models that may be used in age prediction based on cfDNA fragment sizes can include absolute shrinkage and selection operator (LASSO), ridge regression, support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbour algorithm, probably approximately correct learning (PAC) learning, ripple down rules, a knowledge acquisition methodology, symbolic machine learning algorithms, sub symbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm.

As further examples, a model (e.g., a machine learning model) may utilize linear regression, logistic regression, deep recurrent neural network (e.g., long short-term memory, etc.), a hidden Markov model (HMM), linear discriminant analysis (LDA), k-means clustering, density-based spatial clustering of applications with noise (DBSCAN), random forest algorithm, etc. to predict age based on cfDNA fragment sizes. Such a model for predicting age based on cfDNA fragment sizes can be referred to herein as a fragment size clock.

In a particular example, a model (e.g., a LASSO regression model) can be developed for predicting biological age based on the cfDNA fragment sizes. That is, the sizes of the cfDNA fragments corresponding to each dataset can be used as input features in the model for predicting the biological ages of control subjects (e.g., subjects without cancer) in each dataset (e.g., dataset A, dataset B, and dataset C). The model can be trained and tested using each dataset separately.

In some examples, the cfDNA fragment sizes for each control subject in each dataset can be determined using the positions of the ending sequences of each cfDNA fragment with respect to a reference genome (e.g., a human reference genome). For example, the paired-end sequence reads from the whole-genome paired-end sequencing data for each control subject in each dataset can be aligned to the human reference genome. As a result, positions of the two ends of each cfDNA fragment corresponding to the data can be determined. A distance between the start of a first read in a paired-end sequence read and an end of a second read in the paired-end sequence read can be indicative of the size of the fragment.

After sizes of cfDNA fragments for each control subject are determined, a relative frequency of cfDNA fragments of each size of set of sizes for each control subject can be determined. Each size in the set of sizes can be a particular size (e.g., 100 base pairs (bp), 150 bp, 200 bp, etc.) or each size in the set of sizes can be a size range (e.g., 0-100 bp, 101-200 bp, 201-300 bp, etc.). The relative frequency can be a proportion of cfDNA fragments for each control subject that have each size.

As another example, a ratio of the amounts (e.g., proportion or raw counts) of cfDNA fragments at each size can be used as the relative frequencies, e.g., as was described for the end motifs. As yet another example, the relative frequency can be a ranking of the raw counts, ratio, or proportion of cfDNA fragments at each size (e.g., size range).

The relative frequencies of a set of sizes (e.g., of 600 different sizes) can be determined and used for the age clock. Thus, in one embodiment, the relative frequencies of cfDNA fragments of each different size (e.g., each size from 1 base pair (bp) to 600 bp) can be used as input features to predict the biological ages of the control subjects. In another embodiment, relative frequencies of a set of size ranges can be used as input features to predict the biological ages of the control subjects.

Additionally or alternatively, a set of sizes can include longer fragments beyond 600 bp, such as 700 bp, 800 bp, 900 bp, 1000 bp, 1200 bp, 1500 bp, 2000 bp, 3000 bp, 4000 bp, 5000 bp, or other length values, can be included in the analysis. Thus, there can be sizes in a set of sizes that are greater than 600 bp or sizes ranges in the set of sizes that include values greater than 600 bp. Additionally, any number of different sizes can be used in a set of sizes for the analysis. For example, there may be 5, 10, 20, 30, 40, 50, 60, 70, 80, 100, 200, 300, 400, 500, 600, 700, etc. different sizes used. Moreover, in some examples, a size window associated with each size in a set of sizes can be greater than one. In such examples, each size in the set of sizes can be associated with a size range. For example, a size window may be 50 bp and the corresponding size ranges may include 0-50 bp, 51-100 bp, 101-150 bp, 151-200 bp, 201-250 bp, 251-300 bp, 301-350 bp, 351-400 bp, 401-450 bp, 451-500 bp, 501-550 bp, and 551-600 bp. In other examples, the window sizes can be 2, 5, 10, 20, 30, 40, 50, 100, 200, 300 bp, 400, 500, or other values. Additionally, in some examples, the size windows can be overlapped and/or have varying sizes. Further, in some examples, a set of sizes may not be consecutive. For example, the set of sizes can include 0-50 bp, 60-100 bp, 115-200 bp, etc.

Once the relative frequencies of cfDNA fragments for each size of a set of sizes for each control subject are determined, the control subjects of each dataset can be split for training and testing with a ratio of, for example, 4:1. Thus, a training dataset can comprise relative frequencies of different cfDNA fragment sizes for training subjects and a testing dataset can comprise relative frequencies of different cfDNA fragment sizes for testing subjects. As a result, each dataset (dataset A, dataset B, and dataset C) can be split into a training dataset and a testing dataset.

The model can then be trained and verified using each training dataset and the testing dataset for each of dataset A, B, and C respectively. The training can include fitting the model to the training dataset. That is, training can include tuning hyperparameters associated with the model to improve age prediction by the model based on the relative frequencies of cfDNA fragments sizes in the training dataset. After training, the model can be tested by inputting the relative frequencies of the cfDNA fragment sizes from the testing dataset into the trained model. The trained model can then output predicted biological ages for the control subjects in each testing dataset based on the relative frequencies of the cfDNA fragment sizes. The predicted biological ages can then be compared to true chronological ages of the subjects to estimate an accuracy of the trained model.

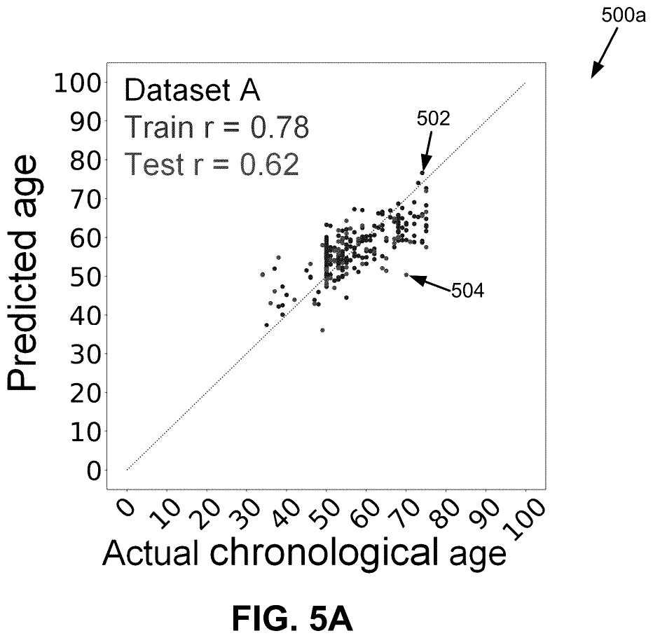

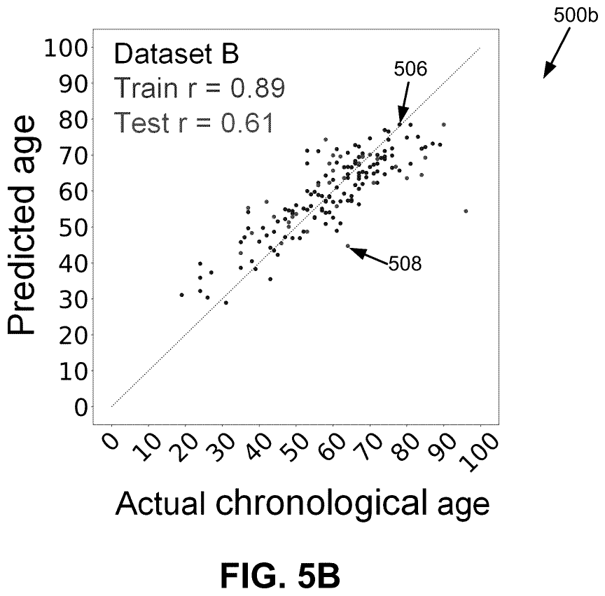

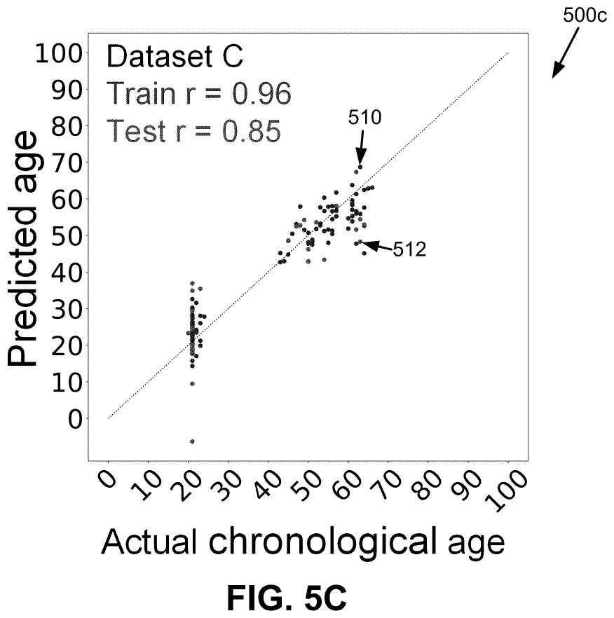

FIG. 5A shows a plot 500a of biological ages predicted based on the relative frequencies of cfDNA fragment sizes against true chronological ages. In particular, plot 500a shows the biological age predictions output by the trained model based on the relative frequencies of cfDNA fragment sizes for the training dataset and the testing dataset associated with dataset A. As an example, point 502 shows a predicted biological age for a subject in the training data set plotted against a true chronological age of the subject, while point 504 shows a predicted biological age for a subject in the testing data set plotted against a true chronological age of the subject

FIG. 5B shows a plot 500b of biological ages predicted based on the relative frequencies of cfDNA fragment sizes against true chronological ages. In particular, plot 500b shows the biological age predictions output by the trained model based on the relative frequencies of cfDNA fragment sizes for the training dataset and the testing dataset associated with dataset B. As an example, point 506 shows a predicted biological age for a subject in the training data set plotted against a true chronological age of the subject, while point 508 shows a predicted biological age for a subject in the testing data set plotted against a true chronological age of the subject

FIG. 5C shows a plot 500c of biological ages predicted based on the relative frequencies of cfDNA fragment sizes against true chronological ages. In particular, plot 500c shows the biological age predictions output by the trained model based on the relative frequencies of cfDNA fragment sizes for the training dataset and the testing dataset associated with dataset C. As an example, point 510 shows a predicted biological age for a subject in the training dataset plotted against a true chronological age of the subject, while point 512 shows a predicted biological age for a subject in the testing data set plotted against a true chronological age of the subject

Each of the plots 500a-c show that the predicted biological ages output by the model can be substantially correlated with the actual chronological ages of the control subjects in dataset A, B, and C. To quantify the correlation between the predicted and true ages depicted in FIGS. 5A-C, a Pearson's correlation coefficient can be computed for each training dataset and each testing dataset. For example, the Pearson's correlation coefficient for the training dataset of dataset A is 0.78 and the Pearson's correlation coefficient for testing dataset of dataset A is 0.62. Additionally, the Pearson's correlation coefficient for the training dataset of dataset B is 0.89 and the Pearson's correlation coefficient for testing dataset of dataset B is 0.61. Moreover, the Pearson's correlation coefficient for the training dataset of dataset C is 0.96 and Pearson's correlation coefficient for the testing dataset of dataset C is 0.85. Thus, in the example, a high concordance between the ages predicted by the end motif clock (e.g., the trained model) and the chronological ages in datasets A, B, and C was found.

### B. Example Method for Age Prediction Based on Fragment Sizes

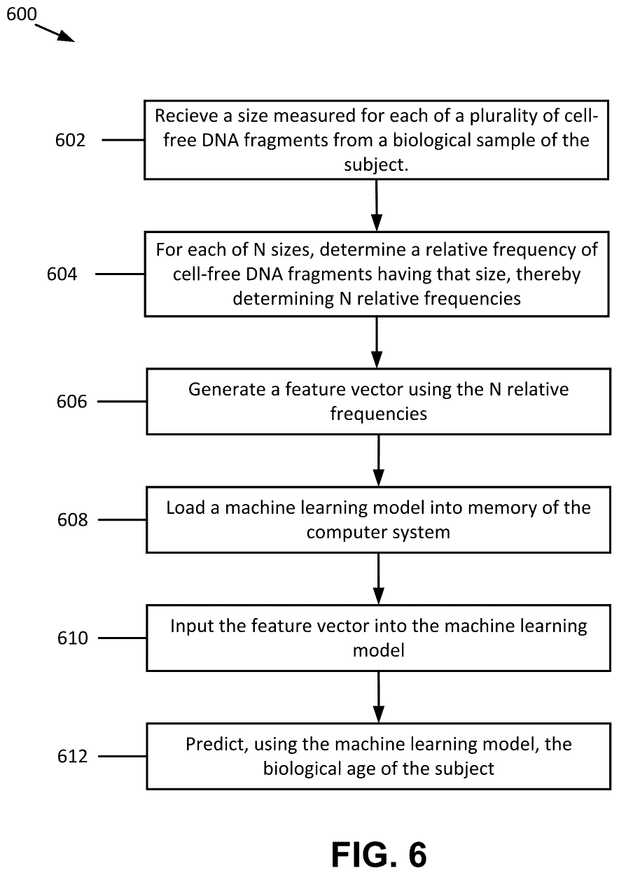

FIG. 6 is a flowchart illustrating a method 600 for measuring a biological age of a subject, according to some embodiments of the present disclosure. Portions or all steps of method 600 can be performed by a computer system, including one or more processors. Method 600 can use a trained ML model that was trained by the computer system or another computer system. The computer system can comprise various devices, e.g., one device that performed the training and another device that uses the trained model.

At block 602, the method 600 can include receiving sizes measured for a plurality of cfDNA fragments from a biological sample of the subject. The biological sample can be any cell-free sample from the subject, e.g., as described herein, such as plasma, urine, saliva, cerebrospinal fluid, pleural fluid, amniotic fluid, peritoneal fluid, ascitic fluid, or the like. Each size may be individually measured for each of the plurality of cell-free DNA fragments. In some examples, the method 600 can further include measuring the size of each of the plurality of cfDNA fragments. The sizes may be measured in aggregate. As an example, electrophoresis may be used to measure an amount the plurality of cfDNA fragments of a particular size. Such captured cfDNA fragments of a particular size can be quantified using an intensity the plurality of cfDNA fragments corresponding to that particular size, such as by using real-time PCR. Thus, in some examples, the intensity can be indicative of relative amount of cfDNA fragments having an estimated size or size range.

Additionally or alternatively, the method 600 can include receiving one or more sequence reads for each cfDNA fragment, and using the one or more sequence reads to determine the size of each cfDNA fragment. As examples, the sequence reads can be generated from paired-end sequencing, single-molecule sequencing, targeted sequencing, or the like, as well as probe-based techniques. The sequence reads can be analyzed, aligned with a reference genome, combined with other data (e.g., paired-end data), or a combination thereof to estimate the size of each cfDNA fragment. In one example, the sequence reads can be paired-end sequence reads, and using the one or more sequence reads to determine the size of the cell-free DNA fragment can include aligning the paired-end sequence reads to a reference sequence. Once aligned, a distance between at least two positions on the reference genome that correspond to each paired-end sequence read can be used to determine the size of each cfDNA fragment.

At block 604, the method 600 can include, for each of M sizes, determining a relative frequency of cell-free DNA fragments having that size, thereby determining M relative frequencies. In some examples, M can be greater than 10. Examples of M can include at least 1, 2, 3, 4, 5, 6, 7, 8, 9, 10, 20, 30, 40, 50, 60, 70, 80, 90, 100, 200, 300, 400, 500, 600, 700, 800, 900, 1000, or other values. Additionally, in some examples, each of the M sizes can be a size range of two or more nucleotides such that M size ranges are used. For example, the size ranges may include a first size of 0-50 nucleotides, a second size of 51-100 nucleotides, a third size of 101-150 nucleotides, a fourth size of 151-200 nucleotides, etc.

A relative frequency can provide a proportion of the plurality of cfDNA fragments that have a size. Alternatively, the relative frequency of cell-free DNA fragments having a size may include a ratio of (1) a first amount of the plurality of cell-free DNA fragments that have the size and (2) a second amount of the plurality of cell-free DNA fragments that have one or more other sizes different than the size. Moreover, in other examples, a relative frequency of a sequence motif includes a ranking of a first amount of the plurality of cell-free DNA fragments that have a size relative to amounts of the plurality of cell-free DNA fragments that have sizes different than the size.

As further examples, the M sizes may include 100 bp, have a lower bound that is equal to or less than 100 b, have an upper bound that is greater than 500 bp, or a combination thereof. Any number of size ranges can be used and the range of values in each size range can differ. For example, the range of values can be greater than or less than 50 nucleotides. The size ranges may also go up to any value (e.g., up to or greater than 600 nucleotides). Additionally, at least two of the M size ranges may overlap. For example, the age ranges can include 0-50 nucleotides, 50-100 nucleotides, 100-150 nucleotides, 150-200 nucleotides, etc. Further, at least two of the M size ranges may not be contiguous. For example, the age ranges can include 0-50 nucleotides, 75-125 nucleotides, 150-300, etc. Alternatively, each of the M sizes can be a specified number of nucleotides (e.g., 2, 5, 10, 20, 30, 40, 50, 100, etc.).

At block 606, the method 600 can include generating a feature vector using the M relative frequencies. The feature vector can include the M relative frequencies of each of the M sizes determined for the cfDNA fragments of the biological sample from the subject. The feature vector can include the M relative frequencies in a structured form that can be ingested (input) into and understood by a machine learning model. As examples, the feature vector can include at least 2, 3, 4, 5, 6, 7, 8, 9, 10, 11, 12, 13, 14, 15, 16, 32, 64, 80, 128, 160, 256, 320, 640, 1,024, 1,280, 2,560, 3,200, and 4,096 features.

At block 608, the method 600 can include loading a machine learning model into memory of the computer system. The machine learning model can be trained using training samples having known chronological ages and measured reference vectors of relative frequencies of the M sizes. In an example, the training samples can be obtained from a training cohort, such as the cohorts (e.g., Dataset A, Dataset B, and Dataset C) described herein. The training cohort can include a known chronological age for each training sample. The training samples can be subjects that do not have a particular pathology. The machine learning model may use clustering, support vector machines, regression, etc.

At block 610, the method 600 can include inputting the feature vector into the machine learning model. That is, the M relative frequencies of the M sizes determined for the cfDNA fragments of the biological sample from the subject can be input into the machine learning model.

At block 612, the method 600 can include predicting, using the machine learning model, the biological age of the subject. Block 612 can be performed in a similar manner as block 414 of method 400.

### V. BIOLOGICAL AGE PREDICTION BASED ON END MOTIFS AND SIZE

Cell-free DNA (cfDNA) fragments exhibit unique characteristics that can provide insight into their origin or underlying biological mechanisms. These characteristics can involve patterns of ending sequences of the cfDNA fragments (i.e., end motifs) and patterns in sizing of the cfDNA fragments. As described above, the characteristics can be used individually to predict a biological age of a subject. The characteristics may also be used in combination to predict biological age. Examples in which end motif and size are used in combination to predict a biological age may include analysis of the ending sequences of cfDNA fragments of different sizes.

### A. Fragmentomic Clock

In some examples, the end motif patterns across different size ranges of cfDNA molecules can be analysed and used to predict biological ages of subjects.

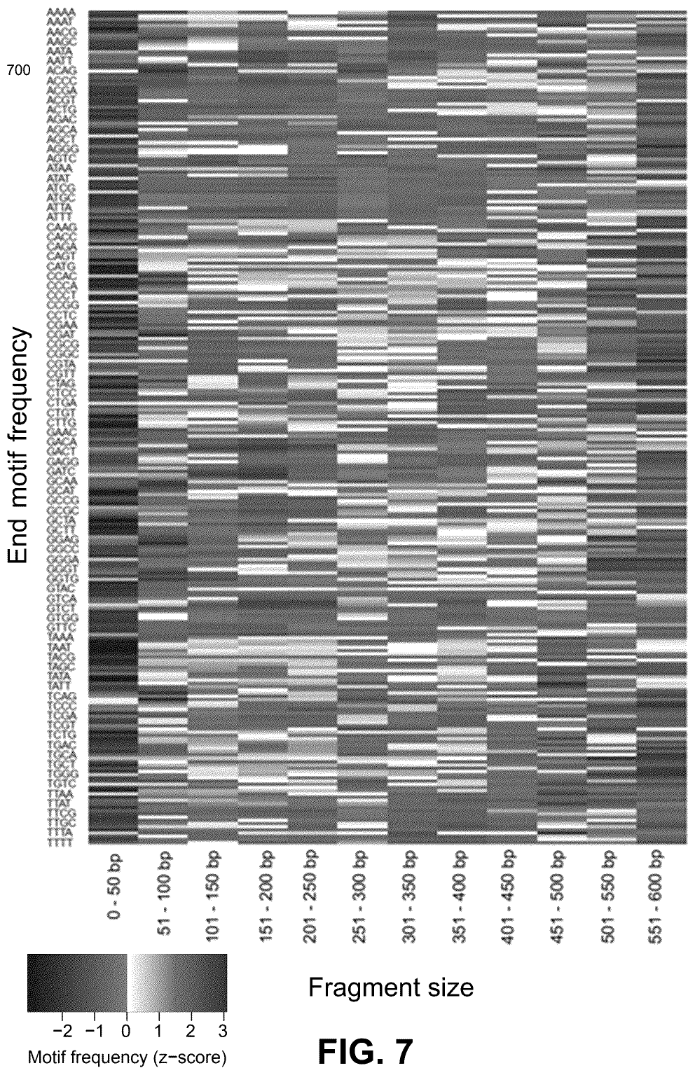

FIG. 7 shows a plot 700 of end motif frequency against fragment size for cfDNA fragments, according to some embodiments of the present disclosure. In particular, the plot 700 shows relative frequencies of 4-mer end motifs for each of twelve size ranges. Thus, the plot 700 shows the relative frequencies of the 4-mer end motifs (i.e., 256 end motifs) within twelve populations of cfDNA molecules having one of the twelve size ranges. Accordingly, the heat map in plot 700 has N (256) by M (12) values. As shown, the cfDNA molecules with different size ranges exhibit different patterns of end motifs. For example, cfDNA molecules within a 301-350 bp size range can be enriched in CCCA end motifs in comparison with the cfDNA molecules within the 51-100 bp size range.

In some examples, a size range of cfDNA molecules being analysed (e.g. from 0 to 600 base pairs (bp)) can be divided into different non-overlapping windows. For example, the windows can have a size of 50 bp and may include 0-50 bp, 51-100 bp, 101-150 bp, 151-200 bp, 201-250 bp, 251-300 bp, 301-350 bp, 351-400 bp, 401-450 bp, 451-500 bp, 501-550 bp, and 551-600 bp. In other examples, the windows can be extended beyond 600 bp, such as 700 bp, 800 bp, 900 bp, 1000 bp, 1200 bp, 1500 bp, 2000 bp, 3000 bp, 4000 bp, 5000 bp, or other length values. Additionally, in other examples, the window sizes can be 2 bp, 5 bp, 10 bp, 20 bp, 30 bp, 40 bp, 50 bp, 100 bp, 200 bp, 300 bp, 400 bp, 500 bp, 1000 bp, or other values. Additionally, in some examples, the windows can be overlapped and/or have varying sizes.

In a particular example, a model (e.g., a LASSO regression model) can be developed for predicting biological age based on the relative frequencies of 4-mer end motifs for various cfDNA fragment sizes. That is, the relative frequencies of 4-mer end motifs for cfDNA fragments of one or more size ranges for each dataset (e.g., Dataset A, Dataset B, and Dataset C) can be used as input features in the model. Thus, the relative frequencies of 4-mer end motifs for cfDNA fragments in each of a set of size ranges can be used to predict the biological ages of control subjects (e.g., subjects without cancer) in each dataset (e.g., dataset A, dataset B, and dataset C). The model can be trained and tested using each dataset separately.

As described with respect to the fragment size clock, the cfDNA fragment sizes for each control subject in each dataset can be determined using the positions of the ends of each cfDNA with reference to a reference genome (e.g., a human reference genome). For example, the paired-end sequence reads from the whole-genome paired-end sequencing data for each control subject in each dataset can be aligned to the human reference genome. As a result, positions of the two ends of each cfDNA fragment corresponding to the data can be determined. A distance between the position of the ends of each cfDNA fragment can be indicative of its size. If the entire cfDNA fragment (molecule) is sequenced, then the size can be determined from the sequence read itself, without any alignment to a reference genome.

In addition to determining a size of each cfDNA fragment corresponding to the datasets, a 4-mer end motif for each cfDNA fragment can be determined. For example, as described with respect to the end motif clock, the first 4-nucleotide sequence on each 5′ fragment end can be referred to as a 5′ 4-mer end motif Thus, once the sequence reads of the paired-end sequencing data are aligned to the reference genome, the smallest coordinate on the reference genome for each sequence read can be defined as the 5′ end. The 4-mer end motif at a 5′ end can then be identified by the four nucleotides in the reference genome (e.g., the nucleotides on the Watson strand of the reference genome) starting from the smallest coordinate. As described above, the 5′ end can be determined for each cfDNA fragment for either or both strands.

Once a size and 4-mer end motif for each cfDNA fragment are identified, a relative frequency of each end motif for each of a set of fragment sizes can be determined. For example, a relative frequency of each 4-mer end motif in cfDNA fragments within the sizes ranges of 0-50 bp, 51-100 bp, 101-150 bp, 151-200 bp, 201-250 bp, 251-300 bp, 301-350 bp, 351-400 bp, 401-450 bp, 451-500 bp, 501-550 bp, and 551-600 bp can be determined. The relative frequencies can therefore be a proportion of cfDNA fragments within each size range with each 4-mer end motif In other examples, the end motifs of cfDNA fragments used for fragmentomic clock can be the first 1-, 2-, 3-, 4-, 5-, 6-, 7-, 8-, 9-, and 10-nucleotide sequence on the 5′ end of cfDNA fragments. Additionally, in other examples other size ranges, a different of size ranges, or longer fragments beyond 600 bp, such as 700 bp, 800 bp, 900 bp, 1000 bp, 1200 bp, 1500 bp, 2000 bp, 3000 bp, 4000 bp, 5000 bp, or other length values, can be used for the fragmentomic clock. For the example in FIG. 7 for 4-mer end motifs (N=256) and M=12 size ranges, a total of 3,072 features (relative frequencies) can be used.

Once the relative frequencies for each control subject are determined, the control subjects of each dataset can be split for training and testing with a ratio of, for example, 4:1. Thus, training dataset can comprise the relative frequencies of 4-mer end motifs for different cfDNA fragment sizes for training subjects and a testing dataset can comprise relative frequencies of 4-mer end motifs for different cfDNA fragment sizes for testing subjects. As a result, each dataset (Dataset A, Dataset B, and Dataset, C) can be split into a training dataset and a testing dataset.

The model can then be trained and verified using each training dataset and the testing dataset respectively. The training can include fitting the model to the training dataset. That is, training can include tuning hyperparameters associated with the model to improve age prediction by the model based on the relative frequencies of cfDNA fragment sizes in the training dataset. After training, the model can be tested by inputting the relative frequencies of the 4-mer end motifs for each of the cfDNA fragment sizes from the testing dataset into the trained model. The trained model can then output predicted biological ages for the control subjects in each testing dataset based on the relative frequencies. The predicted biological ages can then be compared to true chronological ages of the subjects to estimate an accuracy of the trained model.

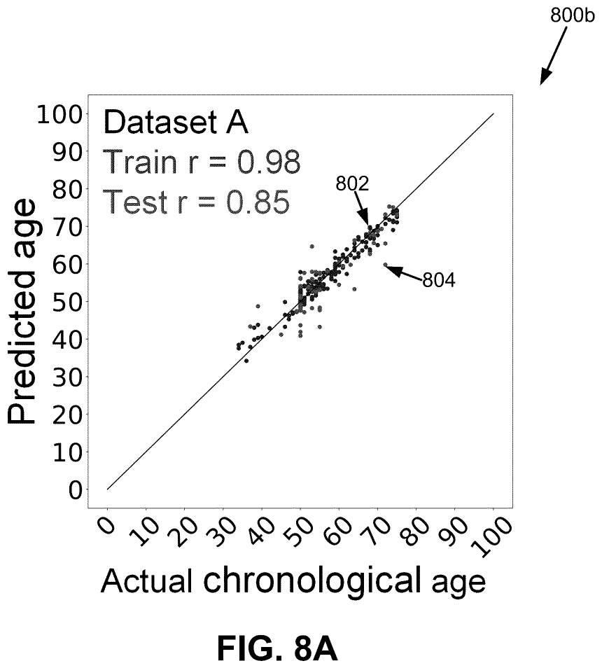

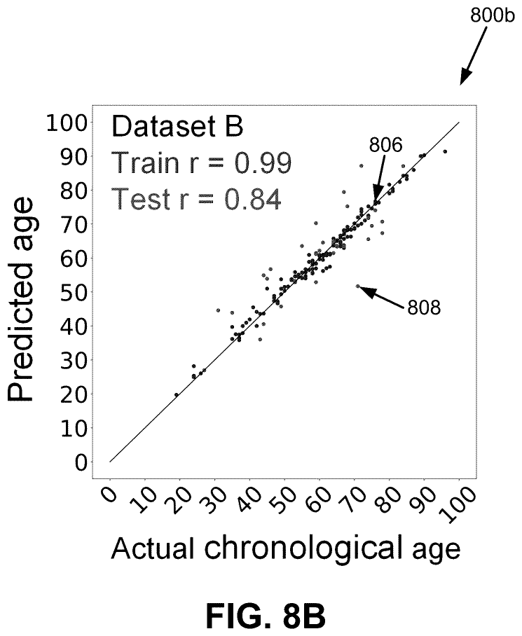

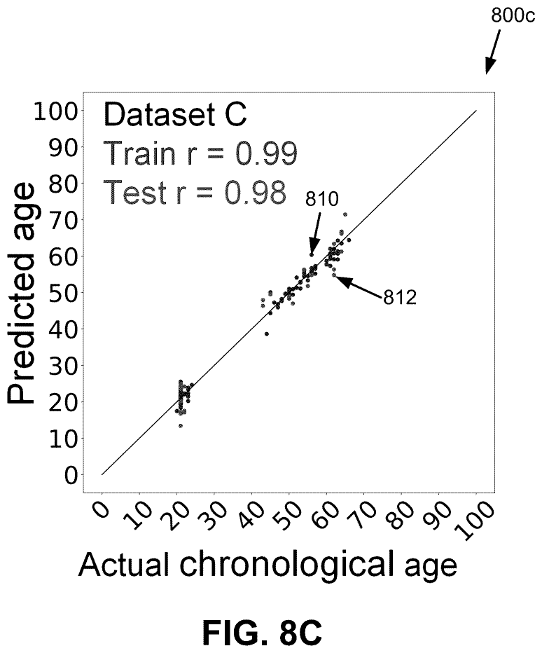

FIG. 8A shows a plot 800a of biological ages predicted based on the relative frequencies of 4-mer end motifs for various cfDNA fragment sizes against true chronological. In particular, plot 800a shows the biological age predictions output by the trained model based on the relative frequencies of 4-mer end motifs for various cfDNA fragment sizes for the training dataset and the testing dataset associated with dataset A. As an example, point 802 shows a predicted biological age for a subject in the training data set plotted against a true chronological age of the subject, while point 804 shows a predicted biological age for a subject in the testing data set plotted against a true chronological age of the subject.

FIG. 8B shows a plot 800b of biological ages predicted based on the relative frequencies of 4-mer end motifs for various cfDNA fragment sizes against true chronological. In particular, plot 800b shows the biological age predictions output by the trained model based on the relative frequencies of 4-mer end motifs for various cfDNA fragment sizes for the training dataset and the testing dataset associated with dataset B. As an example, point 806 shows a predicted biological age for a subject in the training data set plotted against a true chronological age of the subject, while point 808 shows a predicted biological age for a subject in the testing data set plotted against a true chronological age of the subject.

FIG. 8C shows a plot 800c of biological ages predicted based on the relative frequencies of 4-mer end motifs for various cfDNA fragment sizes against true chronological. In particular, plot 800c shows the biological age predictions output by the trained model based on the relative frequencies of 4-mer end motifs for various cfDNA fragment sizes for the training dataset and the testing dataset associated with dataset C. As an example, point 810 shows a predicted biological age for a subject in the training data set plotted against a true chronological age of the subject, while point 812 shows a predicted biological age for a subject in the testing data set plotted against a true chronological age of the subject.

Each of the plots 800a-c show that the predicted biological ages output by the model can be substantially correlated with the actual chronological ages of the control subjects in Dataset A, B, and C. For example, the Pearson's correlation coefficient for the training dataset of Dataset A is 0.98 and the Pearson's correlation coefficient for testing dataset of Dataset A is 0.85. Additionally, the Pearson's correlation coefficient for the training dataset of Dataset B is 0.99 and the Pearson's correlation coefficient for testing dataset of Dataset B is 0.84. Moreover, the Pearson's correlation coefficient for the training dataset of Dataset C is 0.99 and Pearson's correlation coefficient for the testing dataset of Dataset C is 0.98. Thus, in the example, a high concordance between the ages predicted by the fragmentomic clock (e.g., the trained model) and the chronological ages in Datasets A, B, and C was found.

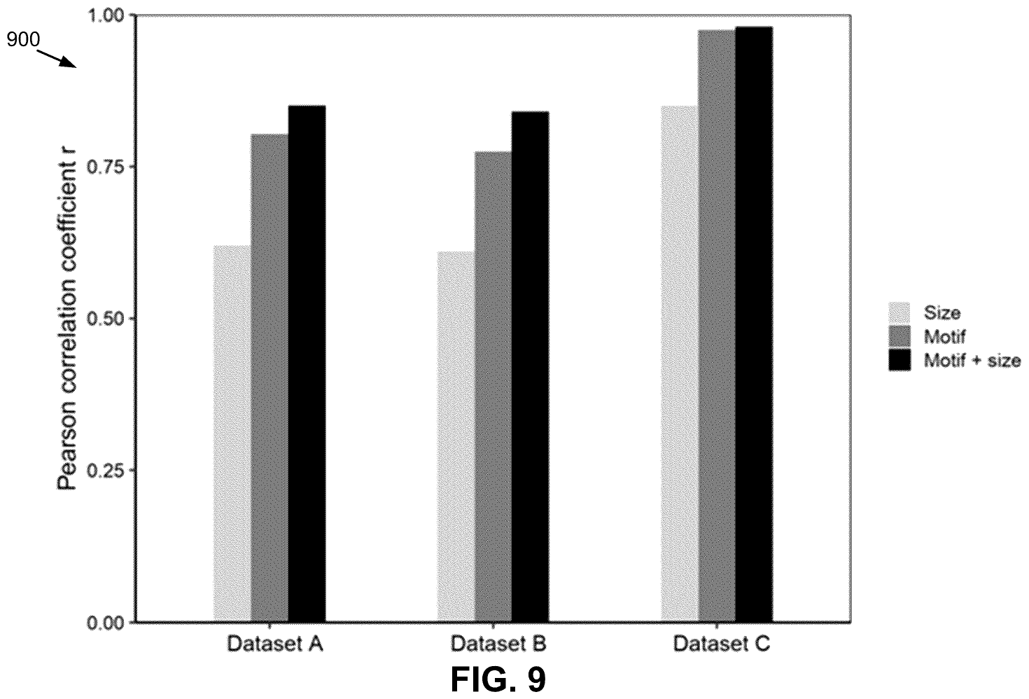

FIG. 9 shows a plot of the Pearson correlation values corresponding to the testing datasets derived from dataset A, dataset B, and dataset C. For each dataset, there are three Pearson correlation values corresponding to the end motif clock, fragment size clock, and a fragmentomic clock. As shown by the Pearson correlation values in the plot 900, compared with using end motif patterns or using fragment sizes, the combined use of end motifs and fragment sizes can enhance an accuracy of biological age prediction.

### B. Example Method for Age Prediction Using End Motifs and Fragment Sizes

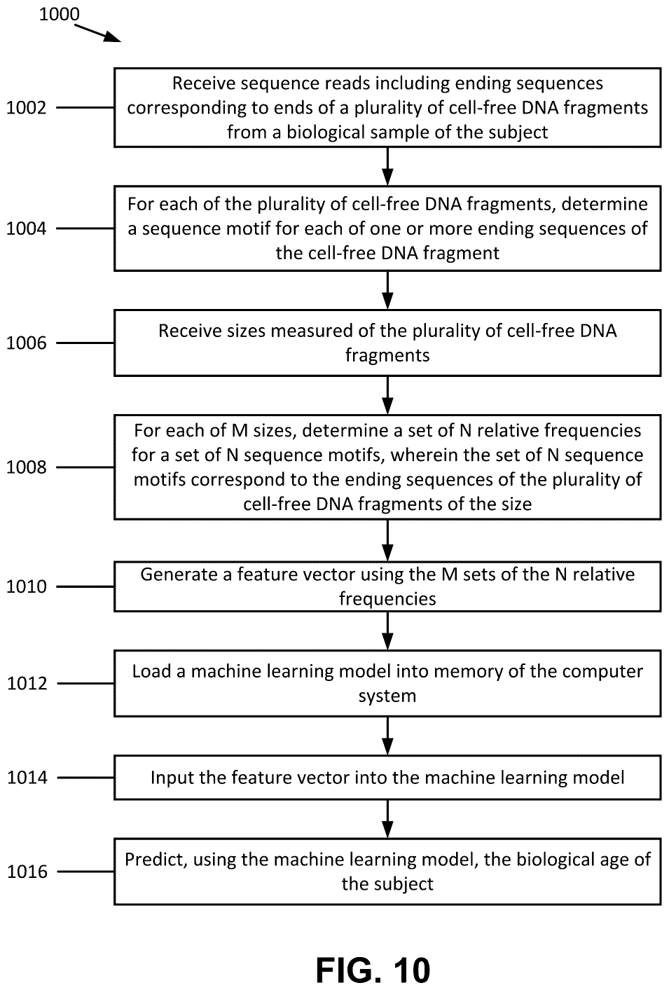

FIG. 10 is a flowchart illustrating a method 1000 for measuring a biological age of a subject, according to some embodiments of the present disclosure. Portions or all steps of method 1000 can be performed by a computer system, including one or more processors. Method 1000 can use a trained ML model that was trained by the computer system or another computer system. The computer system can comprise various devices, e.g., one device that performed the training and another device that uses the trained model.

At block 1002, the method 1000 can include receiving sequence reads including end sequences corresponding to ends of a plurality of cell-free DNA fragments from a biological sample of the subject. Block 1002 can be performed in a similar manner as block 402 of method 400.

At block 1004, the method 1000 can include, for each of the plurality of cell-free DNA fragments, determining a sequence motif for each of one or more ending sequences of the cell-free DNA fragment. Block 1004 can be performed in a similar manner as block 404 of method 400.

At block 1006, the method 1000 can include receiving sizes measured for each of the plurality of cell-free DNA fragments from the biological sample of the subject. Block 1006 can be performed in a similar manner as block 602 of method 600.

At block 1008, the method 1000 can include, for each of M sizes, determine a set of N relative frequencies for a set of N sequence motifs. The set of N sequence motifs can correspond to the ending sequences of the plurality of cell-free DNA fragments of the size. A relative frequency of a sequence motif can provide a proportion of the plurality of cell-free DNA fragments that have an ending sequence corresponding to the sequence motif In some examples, M can be an integer equal to or greater than, e.g., 10, 20, 30, 40, 50, 60, 70, 80, 90, 100, 200, 300, 400, 500, 600, etc., or any integer there between and N can be an integer equal to or greater than, e.g., 16, 32, 64, 70, 80, 90, 100, 110, 120, 140, 150, 160, 170, 180, 190, 200, 210, 220, 230, 240, 250, 256, or any integer there between. In other examples, M can be less than 10 and/or N can be less than 16.

At block 1010, the method 1000 can include generating a feature vector using the M sets of N relative frequencies of the set of N sequence motifs. The feature vector can include the M sets of N relative frequencies of the set of N sequence motifs in a structured form that can be ingested (input) into and understood by a machine learning model. For example, the structured form could be a two dimensional array, such as a matrix. As examples, the feature vector can include at least 16, 32, 64, 80, 128, 160, 256, 320, 640, 1,024, 1,280, 2,560, 3,200, and 4,096 features.

At block 1012, the method 1000 can include loading a machine learning model into memory of the computer system. The machine learning model can be trained using training samples having known chronological ages and measured reference vectors of relative frequencies of the set of N sequence motifs for cell-free DNA fragments of each of the M sizes.

At block 1014, the method 1000 can include inputting the feature vector into the machine learning model. That is, the M sets of N relative frequencies of the set of N sequence motifs determined for the cfDNA fragments of the biological sample from the subject can be input into the machine learning model.

At block 1016, the method 400 can include predicting, using the machine learning model, the biological age of the subject. Block 1016 can be performed in a similar manner as block 414 of method 400.

### VI. TREATMENTS AND FURTHER SCREENING

Responsive to a classification of a pathology or a fractional concentration of clinically-relevant DNA, various actions might be performed, e.g., physical screening steps or treatment(s).

### A. Further Screening Modalities

Based on any classification, e.g., regarding a pathology or fractional concentration of clinically-relevant DNA, the subject can be referred for one or more additional screening modalities, e.g. biopsies (tissue or cell-free, such as liquid or stool) or imaging such as using chest X ray, ultrasound, computed tomography, magnetic resonance imaging, or positron emission tomography. Such screening may be performed for cancer. In this manner, an individual may only be subjected to such screening when (responsive to) there is a high likelihood of the pathology being present, thereby reducing costs, side effects (e.g., radiation exposure), time expenditure of doctor and patients, etc. Additionally, the classification of a pathology (e.g., detection, stage, etc.) can be used to determine a schedule for performing screening modalities, e.g., specifying a frequency for performing the screening modality. The further screening can be performed within a specified amount of time from when the classification is determined, e.g., one day, one week, or one month. The one or more additional screening modalities can be for a particular cancer type, e.g., a particular tissue type, such as imaging a particular organ.

### B. Treatment Selection

Various embodiments of the present disclosure can accurately predict disease relapse, occurrence, and/or severity thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, transplantation, hyperthermia, photodynamic therapy, gene therapy, cell therapy, antibiotics, histotripsy, sound waves, cryoablation, radiofrequency ablation, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Various embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference

### VII. EXAMPLE SYSTEMS

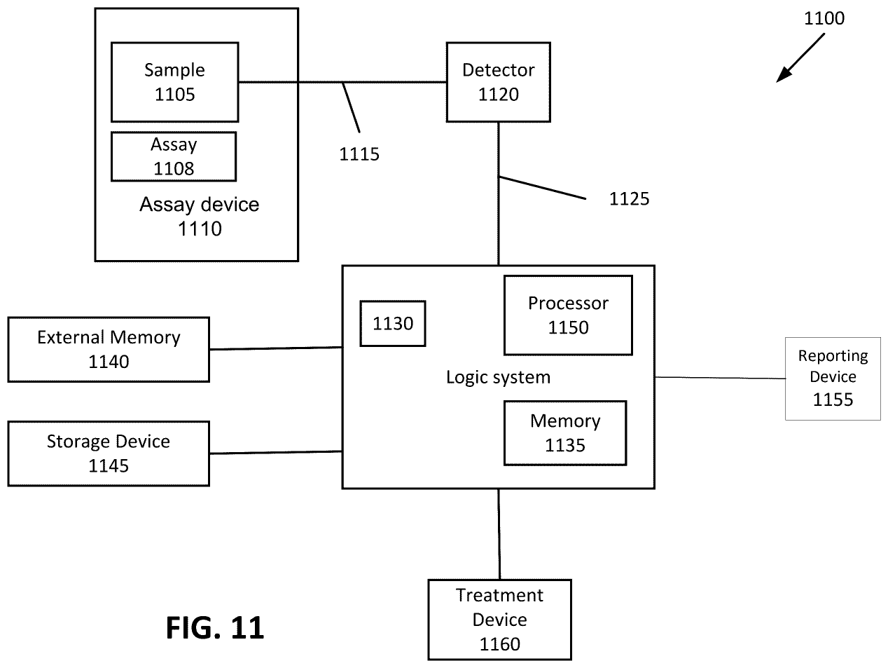

FIG. 11 illustrates a measurement system 1100 according to an embodiment of the present disclosure. The system as shown includes a sample 1105, such as cell-free nucleic acid molecules (e.g., DNA and/or RNA) within an assay device 1110, where an assay 1108 can be performed on sample 1105. For example, sample 1105 can be contacted with reagents of assay 1108 to provide a signal (e.g., an intensity signal) of a physical characteristic 1115 (e.g., sequence information of a cell-free nucleic acid molecule). An example of an assay device can be a flow cell that includes probes and/or primers of an assay or a tube through which a droplet moves (with the droplet including the assay). Physical characteristic 1116 (e.g., a fluorescence intensity, a voltage, or a current), from the sample is detected by detector 1120. Detector 1120 can take a measurement at intervals (e.g., periodic intervals) to obtain data points that make up a data signal. In one embodiment, an analog-to-digital converter converts an analog signal from the detector into digital form at a plurality of times.

Assay device 1110 and detector 1120 can form an assay system, e.g., a PCR system or a sequencing system that performs sequencing according to embodiments described herein. A data signal 1125 is sent from detector 1120 to logic system 1130. As an example, data signal 1125 can be used to determine sequences and/or locations in a reference genome of nucleic acid molecules (e.g., DNA and/or RNA). Data signal 1125 can include various measurements made at a same time, e.g., different colors of fluorescent dyes or different electrical signals for different molecule of sample 1105, and thus data signal 1125 can correspond to multiple signals. Data signal 1125 may be stored in a local memory 1135, an external memory 1140, or a storage device 1145. The assay system can be comprised of multiple assay devices and detectors.

Logic system 1130 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1130 and the other components may be part of a stand-alone or network connected computer system, or they may be directly attached to or incorporated in a device (e.g., a sequencing device) that includes detector 1120 and/or assay device 1110. Logic system 1130 may also include software that executes in a processor 1150. Logic system 1130 may include a computer readable medium storing instructions for controlling measurement system 1100 to perform any of the methods described herein. For example, logic system 1130 can provide commands to a system that includes assay device 1110 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 1100 may also include a treatment device 1160, which can provide a treatment to the subject. Treatment device 1160 can determine a treatment and/or be used to perform a treatment. Examples of such treatment can include surgery, radiation therapy, chemotherapy, immunotherapy, targeted therapy, hormone therapy, and stem cell transplant. Logic system 1130 may be connected to treatment device 1160, e.g., to provide results of a method described herein. The treatment device may receive inputs from other devices, such as an imaging device and user inputs (e.g., to control the treatment, such as controls over a robotic system).

Measurement system 1100 may also include a reporting device 1155, which can present results of any of the methods describe herein, e.g., as determined using the measurement system. Reporting device 1155 can be in communication with a reporting module within logic system 1130 that can aggregate, format, and send a report to reporting device 1155. The reporting module can present information determined using any of the method described herein. The information can be presented by reporting device 1155 in any format that can be recognized and interpreted by a user of the measurement system 1100. For example, the information can be presented by reporting device 1155 in a displayed, printed, or transmitted format, or any combination thereof.

Any of the computer systems mentioned herein may utilize any suitable number of subsystems. Examples of such subsystems are shown in FIG. 12 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 12 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. The computations can be performed in parallel by the different processing units and/or different processing threads of a single processing unit. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as solely, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

### VIII. REFERENCES

Partridge, L., J. Deelen, and P. E. Slagboom, Facing up to the global challenges of ageing. Nature, 2018. 561(7721): p. 45-56.
Lowsky, D. J., et al., Heterogeneity in healthy aging. Journals of Gerontology Series A: Biomedical Sciences and Medical Sciences, 2014. 69(6): p. 640-649.
Hannum, G., et al., Genome-wide methylation profiles reveal quantitative views of human aging rates. Molecular cell, 2013. 49(2): p. 359-367.
Horvath, S., DNA methylation age of human tissues and cell types. Genome biology, 2013. 14(10): p. 1-20.
Peters, M. J., et al., The transcriptional landscape of age in human peripheral blood. Nature communications, 2015. 6(1): p. 1-14.
Fleischer, J. G., et al., Predicting age from the transcriptome of human dermal fibroblasts. Genome biology, 2018. 19: p. 1-8.
Hertel, J., et al., Measuring biological age via metabonomics: the metabolic age score. Journal of proteome research, 2016. 15(2): p. 400-410.
Robinson, O., et al., Determinants of accelerated metabolomic and epigenetic aging in a UK cohort. Aging Cell, 2020. 19(6): p. e13149.
Lehallier, B., et al., Undulating changes in human plasma proteome profiles across the lifespan. Nature medicine, 2019. 25(12): p. 1843-1850.
Oh, H. S.-H., et al., Organ aging signatures in the plasma proteome track health and disease. Nature, 2023. 624(7990): p. 164-172.
Lo, Y. D., et al., Epigenetics, fragmentomics, and topology of cell-free DNA in liquid biopsies. Science, 2021. 372(6538): p. eaaw3616.
Chan, R. W., et al., Plasma DNA profile associated with DNASE1L3 gene mutations: clinical observations, relationships to nuclease substrate preference, and in vivo correction. Am J Hum Genet, 2020. 107(5): p. 882-894.
Ding, S. C., et al., Jagged ends on multinucleosomal cell-free DNA serve as a biomarker for nuclease activity and systemic lupus erythematosus. Clin Chem, 2022. 68(7): p. 917-926.
Jiang, P., et al., Plasma DNA end-motif profiling as a fragmentomic marker in cancer, pregnancy, and transplantation. Cancer Discov, 2020. 10(5): p. 664-673.
Yu, S. C., et al., Single-molecule sequencing reveals a large population of long cell-free DNA molecules in maternal plasma. Proc Natl Acad Sci USA, 2021. 118(50): p. e2114937118.
Choy, L. L., et al., Single-molecule sequencing enables long cell-free DNA detection and direct methylation analysis for cancer patients. Clin Chem, 2022. 68(9): p. 1151-1163.
Yu, S. C., et al., Comparison of single molecule, real-time sequencing and nanopore sequencing for analysis of the size, end-motif, and tissue-of-origin of long cell-free DNA in plasma. Clin Chem, 2023. 69(2): p. 168-179.
Yu, S. C., et al., Combined count- and size-based analysis of maternal plasma DNA for noninvasive prenatal detection of fetal subchromosomal aberrations facilitates elucidation of the fetal and/or maternal origin of the aberrations. Clin Chem, 2017. 63(2): p. 495-502.
Mouliere, F., et al., Enhanced detection of circulating tumor DNA by fragment size analysis. Sci Transl Med, 2018. 10(466): p. eaat4921.
Cristiano, S., et al., Genome-wide cell-free DNA fragmentation in patients with cancer. Nature, 2019. 570(7761): p. 385-389.
Hudecova, I., et al., Characteristics, origin, and potential for cancer diagnostics of ultrashort plasma cell-free DNA. Genome Res, 2022. 32(2): p. 215-227.
Esfahani, M. S., et al., Inferring gene expression from cell-free DNA fragmentation profiles. Nat Biotechnol, 2022. 40(4): p. 585-597.
Snyder, M. W., et al., Cell-free DNA comprises an in vivo nucleosome footprint that informs its tissues-of-origin. Cell, 2016. 164(1): p. 57-68.
Ulz, P., et al., Inference of transcription factor binding from cell-free DNA enables tumor subtype prediction and early detection. Nat Commun, 2019. 10(1): p. 4666.
Zhu, G., et al., Tissue-specific cell-free DNA degradation quantifies circulating tumor DNA burden. Nat Commun, 2021. 12(1): p. 2229.
De Sarkar, N., et al., Nucleosome patterns in circulating tumor DNA reveal transcriptional regulation of advanced prostate cancer phenotypes. Cancer Discov, 2023. 13(3): p. 632-653.
Sun, K., et al., Orientation-aware plasma cell-free DNA fragmentation analysis in open chromatin regions informs tissue of origin. Genome Res, 2019. 29(3): p. 418-427.
Mathios, D., et al., Detection and characterization of lung cancer using cell-free DNA fragmentomes. Nature communications, 2021. 12(1): p. 5060.
Jiang, P., et al., Plasma DNA end-motif profiling as a fragmentomic marker in cancer, pregnancy, and transplantation. Cancer Discovery, 2020. 10(5): p. 664-673.

What is claimed is:

1. A method for measuring a biological age of a subject, the method comprising performing by a computer system:
receiving sequence reads including ending sequences corresponding to ends of a plurality of cell-free DNA fragments from a biological sample of the subject;
for each of the plurality of cell-free DNA fragments, determining a sequence motif for each of one or more ending sequences of the cell-free DNA fragment, thereby determining a set of ending sequences;
determining N relative frequencies of a set of N sequence motifs corresponding to the set of ending sequences of the plurality of cell-free DNA fragments, N being an integer equal to or greater than 16;
generating a feature vector using the N relative frequencies;
loading a machine learning model into memory of the computer system, the machine learning model being trained using training samples having known chronological ages and having measured reference vectors of the set of N sequence motifs of cell-free DNA fragments;
inputting the feature vector into the machine learning model; and
predicting, using the machine learning model, the biological age of the subject.

2. The method of claim 1, wherein the set of N sequence motifs include M base positions, wherein the set of N sequence motifs include all combinations of M bases, and wherein M is an integer equal to or greater than two.

3. The method of claim 1, further comprising:
analyzing the plurality of cell-free DNA fragments from the biological sample to obtain the sequence reads.

4. The method of claim 3, wherein the analyzing includes detecting signals measured from the plurality of cell-free DNA fragments.

5. The method of claim 3, wherein analyzing the plurality of cell-free DNA fragments includes preparing a sequencing library from the plurality of cell-free DNA fragments and sequencing the sequency library.

6. The method of claim 1, wherein the relative frequency of a sequence motif includes a proportion of all the set of ending sequences that have the sequence motif.

7. The method of claim 1, wherein the relative frequency of a sequence motif includes a ratio of (1) a first amount of the set of ending sequences that have the sequence motif and (2) a second amount of the set of ending sequences that have one or more other sequence motifs different than the sequence motif.

8. The method of claim 1, wherein the relative frequency of a sequence motif includes a ranking of a first amount of the set of ending sequences that have the sequence motif relative to amounts of the set of ending sequences that have other sequence motifs different than the sequence motif.

9. The method of claim 1, further comprising:
receiving sizes measured of the plurality of cell-free DNA fragments, wherein the N relative frequencies for a first set of N relative frequencies for a first size of M sizes; and
determining other sets of N relative frequencies for other sizes of the M sizes, thereby determining M sets of N relative frequencies wherein the feature vector is generated using the M sets of N relative frequencies of the set of N sequence motifs.

10. A method for measuring a biological age of a subject, the method comprising performing by a computer system:
receiving sizes measured for a plurality of cell-free DNA fragments from a biological sample of the subject;
for each size of M sizes, determining a relative frequency of cell-free DNA fragments having that size, thereby determining M relative frequencies;
generating a feature vector using the M relative frequencies;
loading a machine learning model into memory of the computer system, the machine learning model being trained using training samples having known chronological ages and measured reference vectors of relative frequencies of the M sizes;
inputting the feature vector into the machine learning model; and
predicting, using the machine learning model, the biological age of the subject.

11. The method of claim 10, wherein the relative frequency of cell-free DNA fragments having a size includes a proportion of all the plurality of cell-free DNA fragments that have the size.

12. The method of claim 10, wherein the relative frequency of cell-free DNA fragments having a size includes a ratio of (1) a first amount of the plurality of cell-free DNA fragments that have the size and (2) a second amount of the plurality of cell-free DNA fragments that have one or more other sizes different than the size.

13. The method of claim 10, wherein the relative frequency of a sequence motif includes a ranking of a first amount of the plurality of cell-free DNA fragments that have the size relative to amounts of the plurality of cell-free DNA fragments that have sizes different than the size.

14. The method of claim 10, wherein a size is individually measured for each of the plurality of cell-free DNA fragments.

15. The method of claim 10, wherein M is an integer greater than 10.

16. The method of claim 10, further comprising:
measuring the sizes of the plurality of cell-free DNA fragments from the biological sample.

17. The method of claim 10, wherein measuring the sizes of the plurality of cell-free DNA fragments uses electrophoresis.

18. The method of claim 10, wherein measuring the sizes of the plurality of cell-free DNA fragments includes:
receiving one or more sequence reads of a cell-free DNA fragment; and
using the one or more sequence reads to determine the size of the cell-free DNA fragment.

19. The method of claim 10, wherein the one or more sequence reads include paired-end sequence reads, and wherein using the one or more sequence reads to determine the size of the cell-free DNA fragment includes aligning the paired-end sequence reads to a reference sequence.

20. The method of claim 10, wherein each of the M sizes is a size range of two or more nucleotides such that M size ranges are used.

21. The method of claim 20, wherein at least two of the M size ranges overlap.

22. The method of claim 10, wherein each of the M sizes is a specified number of nucleotides.

23. The method of claim 10, wherein one of the M sizes has a lower bound that is equal to or less than 100 bp.

24. The method of claim 10, wherein one of the M sizes includes 100 bp.

25. The method of claim 10, wherein at least one of the M sizes has an upper bound that is greater than 500 bp.

26. The method of claim 10, wherein one of the M sizes includes 500 bp.

27. The method of claim 1, further comprising:
determining a separation value by comparing the predicted biological age to a chronological age of the subject; and
determining a classification of a pathology for the subject based on the separation value.

28. The method of claim 27, wherein determining the classification of the pathology for the subject includes comparing the separation value to a reference value determined from a first cohort of subjects that have a particular classification of the pathology and a second cohort of subjects that do not have the particular classification of the pathology.

29. The method of claim 28, wherein the particular classification is (1) whether the pathology is presence or (2) a severity or stage of the pathology.

30. The method of claim 27, wherein the pathology is cancer.

31. The method of claim 1, wherein the training samples are of subjects that do not have a particular pathology.

32. The method of claim 1, wherein the machine learning model uses clustering, support vector machines, a neural network, or regression.
